(19) United States
(12) Patent Application Publication
(10) Pub. No.: US 2026/0245000 A1
(43) Pub. Date: Aug. 20, 2026

(54) TRACKING AND MONITORING RESOURCE USAGE BY SUBJECTS

(71) Applicant: LifeLens Technologies, Inc., Ivyland, PA (US)

(72) Inventors: Landy Toth, Doylestown, PA (US); Kevin D’Aquilla, Warminster, PA (US)

(21) Appl. No.: 19/161,421

(22) PCT Filed: Mar. 8, 2024
(86) PCT No.: PCT/US2024/019060

§ 371 Date: Sep. 2, 2025

(51) Int. Cl. G06Q 10/0631; G16H 40/67

(52) U.S. Cl. CPC G06Q 10/0631; G16H 40/67

(57) ABSTRACT

An apparatus comprises at least one processing device comprising a processor coupled to a memory. The at least one processing device is configured to obtain resource usage tracking data from one or more sensing devices associated with a subject, the resource usage tracking data characterizing availability of one or more resources utilized by the subject. The at least one processing device is also configured to determine, based at least in part on the received resource usage tracking data, one or more resource usage metrics for the one or more resources, and to generate, based at least in part on the determined one or more resource usage metrics, one or more feedback signals. The at least one processing device is further configured to transmit the generated one or more feedback signals to at least one additional processing device.

### STATEMENT OF GOVERNMENT RIGHTS

This invention was made with government support under Medical Technology Enterprise Consortium (MTEC) Contract No.: 2019-399 awarded by the Defense Health Agency (DHA). The government has certain rights in the invention.

### TECHNICAL FIELD

The present disclosure relates to the field of resource tracking and, more particularly, to devices and systems for tracking usage of resources expended by subjects to facilitate management of subjects.

### BACKGROUND

Monitoring the functionality, usage and availability of resources is important for managing subjects performing various activities. For example, monitoring the availability of resources for personnel in military or paramilitary environments is important to ensure the success of activities performed by the personnel, safety of the personnel, performing review of the activities performed by the personnel, etc. Personnel in these and other environments may use various types of resources, including disposable resources (e.g., weaponry, ammunition, medical equipment, medicine, food, water, etc.). Tracking the amounts of such resources which have been expended is critical for improving the likelihood of successful operations, and safety of various personnel performing different activities. Moreover, collective information may provide value both for real-time decision-making and retrospective analysis. As the proliferation of mobile and remote medicine and other activity increases, simplified and unobtrusive means for tracking the usage and availability of resources becomes more important. More reliable, redundant and user friendly systems are needed that can provide valuable personnel data even when operating with limited supervision, expert input, or use manipulation, including in remote locations were power and/or local area networks are not readily available.

### SUMMARY

One illustrative, non-limiting objective of this disclosure is to provide systems, devices, and methods for monitoring and tracking resource data associated with supplies carried by one or more subjects. Another illustrative, non-limiting objective is to utilize data collected from subjects to provide feedback to inform subjects of supply status. Yet another illustrative, non-limiting objective is to provide systems, devices, and methods for monitoring inventory of subjects at various remote locations, and for utilizing collected data to compare performance of different subjects.

The above illustrative, non-limiting objectives are wholly or partially met by devices, systems, and methods according to the appended claims in accordance with the present disclosure. Features and aspects are set forth in the appended claims, in the following description, and in the annexed drawings in accordance with the present disclosure.

In one embodiment, an apparatus comprises at least one processing device comprising a processor coupled to a memory. The at least one processing device is configured to obtain resource usage tracking data from one or more sensing devices associated with a subject, the resource usage tracking data characterizing availability of one or more resources utilized by the subject. The at least one processing device is also configured to determine, based at least in part on the received resource usage tracking data, one or more resource usage metrics for the one or more resources, and to generate, based at least in part on the determined one or more resource usage metrics, one or more feedback signals. The at least one processing device is further configured to transmit the generated one or more feedback signals to at least one additional processing device.

At least one of the one or more sensing devices may be part of one or more wearable computing devices associated with the subject.

At least one of the one or more sensing devices may be attached to at least one of: a resource supply device associated with at least one of the subject and a piece of equipment utilized by the subject; and the piece of equipment utilized by the subject. The piece of equipment may be configured to consume the one or more resources.

The one or more resources may comprise ammunition, the resource supply device may comprise an ammunition supply device, and the piece of equipment may comprise a weapon configured to utilize the ammunition.

The one or more resources may comprise medical supplies, the resource supply device may comprise a medical supply container, and the piece of equipment may comprise a medical device configured to consume the medical supplies.

The one or more resources may comprise at least one of water and food rations.

The one or more resource usage metrics may comprise at least one of a quantity of the one or more resources available to the subject, a rate of consumption of the one or more resources by the subject, and a change in the rate of consumption of the one or more resources by the subject.

The at least one additional processing device may comprise a stimulating device associated with the subject, the one or more feedback signals instructing the stimulating device to apply a stimulus to the subject to communicate to the subject the determined one or more resource usage metrics.

The at least one additional processing device may comprise a third-party network managing a supply of the one or more resources utilized by the subject, and the one or more feedback signals may instruct the third-party network to at least one of order an additional quantity of the one or more resources utilized by the subject and deliver the additional quantity of the one or more resources utilized by the subject.

The one or more sensing devices associated with the subject may be part of a body area network associated with the subject, the body area network comprising at least one of one or more physiologic monitoring devices and one or more location tracking devices. Determining the one or more resource usage metrics for the one or more resources may be further based at least in part on at least one of physiologic monitoring data obtained from the one or more physiologic monitoring devices and location data obtained from the one or more location tracking devices.

Determining the one or more resource usage metrics may comprise predicting a change in consumption of the one or more resources based at least in part on at least one of the physiologic monitoring data and the location data. The predicted change in the consumption of the one or more resources may be based at least in part on movement of the subject from a first location towards a second location. The second location may have at least one of different environmental conditions than the first location and different availability of resource re-supply depots. The predicted change in the consumption of the one or more resources may be based at least in part on detecting a change in a health of the subject from a first health state to a second health state.

The generated one or more feedback signals may instruct the subject to move from a first location to a second location, the second location being associated with at least one of a resource re-supply depot and an additional subject having a quantity of the one or more resources available for transfer to the subject.

The at least one processing device may be part of a host device configured to manage a network comprising the one or more sensing devices, and the resource usage tracking data may be obtained based at least in part on monitoring pairing and unpairing of the one or more sensing devices with the network managed by the host device.

In another embodiment, a method comprises obtaining, at a host device, resource usage tracking data from one or more sensing devices associated with a subject, the resource usage tracking data characterizing availability of one or more resources utilized by the subject. The method also comprises determining, at the host device based at least in part on the received resource usage tracking data, one or more resource usage metrics for the one or more resources and generating, at the host device based at least in part on the determined one or more resource usage metrics, one or more feedback signals. The method further comprises transmitting, from the host device to at least one additional device, the generated one or more feedback signals.

In another embodiment, a computer program product comprises a non-transitory processor-readable storage medium having stored therein executable program code which, when executed, causes at least one processing device to obtain resource usage tracking data from one or more sensing devices associated with a subject, the resource usage tracking data characterizing availability of one or more resources utilized by the subject. The executable program code, when executed, also causes the at least one processing device to determine, based at least in part on the received resource usage tracking data, one or more resource usage metrics for the one or more resources and to generate, based at least in part on the determined one or more resource usage metrics, one or more feedback signals. The executable program code, when executed, further causes the at least one processing device to transmit the generated one or more feedback signals to at least one additional processing device.

### BRIEF DESCRIPTION OF THE DRAWINGS

Several aspects of the disclosure can be better understood with reference to the following drawings. In the drawings, like reference numerals designate corresponding parts throughout the several views.

FIG. 1 illustrates aspects of a modular resource monitoring system, according to an embodiment of the invention.

FIGS. 2A-2C illustrate a modular resource monitoring system, according to an embodiment of the invention.

FIGS. 3A-3E illustrate a wearable sensor system configured for monitoring and modeling resource usage tracking data, according to an embodiment of the invention.

FIG. 4 shows a resource monitoring system including sensor and feedback modules coupled to a subject as well as equipment and a resource supply associated with the subject, according to an embodiment of the invention.

FIG. 5 illustrates a process flow for resource usage tracking, according to an embodiment of the invention.

### DETAILED DESCRIPTION

Particular embodiments of the present disclosure are described herein below with reference to the accompanying drawings; however, the disclosed embodiments are merely examples of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures.

The accompanying drawings illustrate various embodiments of systems, methods, and embodiments of various other aspects of the disclosure. One of ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. It is also noted that components and elements in the figures are not necessarily drawn to scale, emphasis instead being placed upon illustrating principles.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

One illustrative, non-limiting objective of this disclosure is to provide systems, devices, methods, and kits for monitoring and tracking resource data associated with supplies carried by one or more subjects. Another illustrative, non-limiting objective is to utilize data collected from subjects to provide feedback informing subjects of supply status. Yet another illustrative, non-limiting objective is to provide systems, devices, and methods for monitoring inventory of subjects at various remote locations, and for utilizing collected data to compare performance of different subjects.

The above illustrative, non-limiting objectives are wholly or partially met by devices, systems, and methods according to the appended claims in accordance with the present disclosure. Features and aspects are set forth in the appended claims, in the following description, and in the annexed drawings in accordance with the present disclosure.

In the following description, the term “subject” is intended to encompass any individual, soldier, group or any unmanned equipment, weaponry, missiles, projectile systems etc. in association with a military, paramilitary and/or other operative environment. The term “resource” is intended to include any equipment, ammunition and/or supplies associated with subject, which may be utilized during one or more activities or operations in an environment. Supplies may include, for example, medical equipment, medication, water, food and/or any other inventory items carried by the subject.

A resource monitoring system in accordance with the present disclosure is configured to monitor and/or track availability and/or usage of resources by a subject. The resource monitoring system includes one or more sensing devices that are configured for monitoring resources. In some cases, the sensing devices are dedicated for tracking resource usage and/or availability, and are thus referred to as resource sensing devices. Such resource sensing devices may be associated with a resource supply (e.g., which are mounted on resource containers, equipment, etc.). The sensing devices may also or alternatively include devices that are not dedicated for tracking resource usage and/or availability. For example, sensing devices configured for physiologic monitoring, referred to as physiologic sensing devices, may be leveraged to also provide functionality for resource tracking. The term “sensing device” as used herein may thus refer to a device comprising one or more sensors that is configured for resource tracking and/or physiologic monitoring.

A subject may carry and/or operate various types of resources in different environments. For example, resources in a military environment may include weaponry, ammunition, supplies such as food and/or water rations, medical equipment, medicine, etc. In some cases, one or more resource sensing devices may be directly mounted to the resources or resource containers, for example, in a line of sight of a supply of resources. In other cases, one or more of the resource sensing devices may be placed in a vicinity of the supply of resources (e.g., such as acoustic sensing devices capable of detecting discharge of ammunition from weaponry, weight sensors capable of detecting when resources are removed from a container, etc.). In still other cases, resource sensing devices may be both directly mounted on a resource supply and placed in the vicinity of the resource supply. The resource monitoring system may be configured to track the availability of resources, or resource usage, associated with an unmanned tactical subject or system such as a missile or projectile system.

In a resource monitoring system, data collected from sensing devices may be analyzed to provide feedback (e.g., to inform one or more subjects, in real time, of the status of different resources), to evaluate performance (e.g., to compare the performance of different subjects relative to their peers), to predict resource need (e.g., quantities of resources required in future activities or events, for initiating ordering or resupply of resources, etc.), etc.

Various equipment used in different settings may rely on disposable system elements. Consider, for example, a military setting. A soldier may utilize modern firearms having ammunition feeding devices which allow the firearms to discharge multiple times before reloading. Numerous battlefield systems and other equipment may similarly utilize disposable system elements. This includes, for example, medical equipment (e.g., bandages, medications, devices for delivering medications, devices for performing medical procedures, physiologic monitoring devices, etc.), rations (e.g., food, water, etc.), electronic equipment (e.g., batteries or power sources for different devices including communications devices, drones, sensors, etc.), etc. Awareness of the amounts of resources available, the total amounts of resources carried by a subject or group of subjects, etc., can be beneficial for different subjects in the field. Continuing with the example above of firearms used in a military setting, awareness of the amount of ammunition available and the total ammunition carried by each individual soldier and/or a group of soldiers can be beneficial for soldiers in the field. When individual subjects cooperate with others in a team settings (e.g., such as a group of soldiers), resource tracking information can also be useful for team leadership, decision-making, and retrospective analysis.

Some embodiments provide systems, devices, methods and kits for monitoring of subject performance and supply status, such as in a military setting where weapon system conditions and loaded ammunition status may be tracked. Such embodiments enable low-upkeep lightweight systems for monitoring the supply of resources available for different subjects (e.g., tactical units of one or more soldiers in a military setting).

In some embodiments, a method includes receiving logistical data (e.g., resource usage and/or tracking data) from a plurality of sensors coupled to an operator, analyzing the logistical data to derive one or more parameters or metrics, and transmitting at least one of the logistical data and the derived parameters or metrics to at least one remote receiver for further processing. The remote receiver may comprise a host device that is in communication with different sensing devices comprising the plurality of sensors. The host device may be embodied, for example, as a wearable device associated with a subject, a wireless gateway that is carried by or associated with the subject, etc. In some cases, the sensing devices are dedicated for resource tracking (and thus referred to as “resource sensing devices”) and are exclusively paired to the host device. The host device may assign unique identifiers to the sensing devices. Such dedicated resource sensing devices may also be designed to coexist with other types of sensing devices (e.g., physiologic monitoring devices) that are part of a body area network (BAN) for a subject, with the BAN being controlled by the host device. In some cases, a single device may be configured for both resource tracking (e.g., and thus function as a “resource sensing device”) as well as physiologic monitoring (e.g., and thus function as a “physiologic monitoring device”). The term “sensing device” as used herein may thus refer to a device comprising one or more sensors that is configured for resource tracking and/or physiologic monitoring. The sensing devices may autonomously form an ad-hoc network, and seek an acceptable host device among a set of potential host devices according to pre-registered options.

In some embodiments, the sensing devices comprise individual, unique units configured to attach to resource supply devices. For example, sensing devices may be configured for attachment to different ammunition feeding devices or other equipment or gear carried by a subject. The sensing devices may comprise a solid enclosure, along with a hardware processor, associated memory, radio transceivers, antennas, and power management functionality. A sensing device may also comprise a sealed integral package, where a circuit board is encased in an overmolded material. In some embodiments, sensing devices may be attached to an exterior of an existing resource supply device (e.g., an existing firearm ammunition feeding device). The sensing devices may include an apparatus for determining changes in the amount of resources within a resource supply device (e.g., the number of cartridges contained inside an ammunition feeding device). This apparatus may be based on or utilize piezoelectric, radar, ultrasonic, resistive, magnetic, capacitive, accelerometric, barometric, audio and/or electrooptical sensor modalities). In some embodiments, a sensing device is attached to a feed ramp of a belt-fed firearm. A sensing device may also be attached to soft or hard belt boxes or feed chutes for use with belt-fed firearms.

In some embodiments, a sensing device is configured to attach to the exterior or interior of single- or multi-use low-velocity or rocket-assisted launchers. The sensing device may attach to individual resource feeding devices or pieces of ammunition for use in such systems.

A sensing device may be mounted to receiving hardware by mechanical, magnetic or chemical adhesive means. Sensing devices, in some embodiments, incorporate radio transceivers configured for communication over various types of networks and utilizing different protocols including but not limited to ultrawideband (UWB), Bluetooth, Bluetooth Low Energy (BLE), Long Range (LoRA), Wifi, Near Field Communication (NFC), etc.

The sensing devices may be configured to communicate with one another and/or a host device using end-to-end encryption. The sensing devices may be configured to automatically unpair from the host device after loss of communications. Sensing devices may also unpair responsive to intentional user input, under host device control, based on distance from the host device, etc. Unpairing of a sensing device may be used to signal or communicate to the host device of a need to update a count of available resources (e.g., a carried ammunition count).

Sensing devices in some embodiments may include a battery, where the battery may be recharged. Sensing devices may also or alternatively be directly powered through physical connection, wirelessly or through energy harvesting. Energy harvesting strategies may include energy derived from passive radio frequency energy, solar cells, vibration, chemical propellant, a flow of gas, etc. A sensing device may also or alternatively include a wired connection for communication to an external computing device (e.g., a host device).

In some embodiments, a resource monitoring system is configured to operate in conjunction with or as part of a modular physiologic monitoring system configured to monitor one or more physiologic and/or physical signals, also referred to herein as physiologic parameters, of a subject (e.g., a human subject, a patient, an athlete, a trainer, a soldier or military personnel, an animal such as equine, canine, porcine, bovine, etc.). The modular physiologic monitoring system may include one or more patches, each patch adapted for attachment to the body of the subject (e.g., attachable to the skin thereof, reversibly attachable, adhesively attachable, with a disposable interface and a reusable module, etc.) and/or to resource supply containers, and/or to equipment that is carried by, used by or otherwise associated with the subject. In aspects, the modular physiologic monitoring system may also include one or more modules, configured and dimensioned to mate with corresponding ones of the one or more patches, and to interface with the subject therethrough. One or more of the modules may be configured to convey and/or store one or more physiologic and/or physical signals, signals derived therefrom, and/or metrics derived therefrom obtained via the interface with the subject. One or more of the modules may also or alternatively be configured to act as a resource sensing device of the resource monitoring system, and may be configured to convey and/or store one or more or resource tracking signals, signals derived therefrom, and/or metrics derived therefrom.

Each module may include a power source (e.g., a battery, a rechargeable battery, an energy harvesting transducer, microcircuit, an energy reservoir, a thermal gradient harvesting transducer, a kinetic energy harvesting transducer, a radio frequency energy harvesting transducer, a fuel cell, a biofuel cell, etc.), signal conditioning circuitry, communication circuitry, one or more sensors, or the like, configured to generate one or more signals (e.g., physiologic and/or physical signals, resource tracking signals), stimulus, etc.

One or more of the patches may include one or more interconnects, configured and dimensioned so as to couple with one or more of the modules, said modules including a complementary interconnect configured and dimensioned to couple with the corresponding patch. The patch may include a bioadhesive interface for attachment to the subject, or another type of adhesive configured for attachment to a resource supply container and/or equipment (e.g., clothing, gear, packs, weapons, ammunition, etc.) that is carried by or otherwise associated with the subject, the module retainable against the subject via interconnection with the patch.

In aspects, one or more patches and/or modules may be configured for electrically conducting interconnection, inductively coupled interconnection, capacitively coupled interconnection, with each other. In the case of an electrically conducting interconnect, each patch and module interconnect may include complementary electrically conducting connectors, configured and dimensioned so as to mate together upon attachment. In the case of an inductively or capacitively coupled interconnect, the patch and module may include complementary coils or electrodes configured and dimensioned so as to mate together upon attachment.

Each patch or patch-module pair may be configured as a sensing device to monitor one or more local physiologic and/or physical parameters of the attached subject (e.g., local to the site of attachment, etc.), local environment (e.g., resource tracking), combinations thereof, or the like, and to relay such information in the form of signals to a host device (e.g., via a wireless connection, via a body area network connection, or the like), one or more patches or modules on the subject, or the like. Each patch and/or patch-module pair may also or alternatively be configured as a stimulating device to apply a stimulus to the subject in response to signaling from the host device, the signaling being based on analysis of the physiologic and/or physical parameters of the subject and/or resource tracking signals measured by the sensing device(s).

In aspects, the host device may be configured to coordinate information exchange to/from each module and/or patch or other sensing device, and to generate one or more physiologic signals, physical signals, environmental signals, kinetic signals, diagnostic signals, alerts, reports, recommendation signals, commands, resource tracking signals, combinations thereof, or the like for the subject, a user, a network, an electronic health record (EHR), a database (e.g., as part of a data management center, an EHR, a social network, etc.), a processor, combinations thereof, or the like. In aspects, the host device may include features for recharging and/or performing diagnostic tests on one or more of the modules. In aspects, a host device in accordance with the present disclosure may be integrated into a bedside alarm clock, housed in an accessory, within a purse, a backpack, a wallet, or may be included in a mobile computing device, a smartphone, a tablet computer, a pager, a laptop, a local router, a data recorder, a network hub, a server, a secondary mobile computing device, a repeater, a combination thereof, or the like.

In aspects, a system in accordance with the present disclosure may include a plurality of substantially similar modules (e.g., generally interchangeable modules, but with unique identifiers), for coupling with a plurality of patches, each patch, optionally different from the other patches in the system (e.g., potentially including alternative sensors, sensor types, sensor configurations, electrodes, electrode configurations, etc.). Each patch may include an interconnect suitable for attachment to an associated module. Upon attachment of a module to a corresponding patch, the module may validate the type and operation of the patch to which it has been mated. In aspects, the module may then initiate monitoring operations (e.g., including resource tracking) on the subject via the attached patch, communicate with one or more other patches on the subject, a hub, etc. The data collection from each module may be coordinated through one or more modules and/or with a host device in accordance with the present disclosure. The modules may report a timestamp along with the data in order to synchronize data collection across multiple patch-module pairs on the subject, between subjects, etc. Thus, if a module is to be replaced, a hot swappable replacement (e.g., replacement during a monitoring procedure) can be carried out easily by the subject, a caregiver, practitioner, etc., during the monitoring process. Such a configuration may be advantageous for performing redundant, continuous monitoring of a subject, and/or to obtain spatially relevant information from a plurality of locations on the subject during use.

One or more devices in the network may include a time synchronization service, the time synchronization service configurable so as to periodically align the local time sources of each device to those of each of the other devices in the network. In aspects, the time synchronization may be performed every second, every ten seconds, every thirty seconds, every minute, or the like. In aspects, one or more local devices may be coupled to an external time source such as an Internet accessible time protocol, or a geolocation-based time source. Such information may be brought into the network so as to help align a global time reference for devices in the network. Such information may propagate through the network devices using the time synchronization service.

In a time aligned configuration, one or more metrics measured from a subject in connection with one or more devices in the network may be time aligned with one or more metrics from a different subject in the network. As such, events that can simultaneously affect multiple subjects can be registered and higher level event classification algorithms are configured so as to generate an appropriate alert based on the metrics measured.

In aspects, an event may include a loud audible event, or a physiological response to an event, the event classification algorithm is configured so as to increase the priority of an alert if the number of subjects affected by the event increases beyond a set number.

In aspects the modules and/or patches may include corresponding interconnects for coupling with each other during use. The interconnects may include one or more connectors, configured such that the modules and patches may only couple in a single unique orientation with respect to each other. In aspects, the modules may be color coded by function. A temporary stiffening element attached to a patch may include instructions, corresponding color coding, etc., so as to assist a user or subject with simplifying the process of monitoring.

In addition to physiologic monitoring and/or resource tracking, one or more patches and/or modules may be used to provide a stimulus to the subject, as will be described in further detail below.

According to aspects there is provided a device (e.g., a module in accordance with the present disclosure) for monitoring physiologic, physical, and/or electrophysiological signals from a subject, and/or resource tracking signals for monitoring resources associated with the subject. The module may include a housing, a printed circuit board (PCB) including one or more microcircuits, and an interconnect configured for placement of the device onto a subject interface (e.g., a patch in accordance with the present disclosure). The PCB may constitute at least a portion of the housing in some embodiments. The module may include a three-dimensional antenna coupled to the microcircuits (e.g., coupled with a transceiver, transmitter, radio, etc., included within the microcircuits). In aspects, the antenna may be printed onto or embedded into the housing. In aspects, the antenna may be printed on an interior wall of or embedded into the housing, the circuit board providing a ground plane for the antenna. In aspects, the housing may be shaped like a dome and the antenna may be patterned into a spiraling helix centered within the dome.

In aspects, a module in accordance with the present disclosure may include a sensor coupled with one or more of the microcircuits, the sensor configured to interface with the subject, and/or resource supply containers or other equipment associated with the subject, upon attachment of the module to the patch. The module may include one or more sensors and/or microelectronics configured to interface with one or more sensors included on a corresponding patch and/or resource supply containers and/or other equipment associated with the subject. In aspects, one or more of the sensors may include an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, a vibration sensor, a weight sensor, a combination thereof, or the like.

In aspects, the module may be hermetically sealed. The module and/or patch may include a gasket coupled to the circuit board or the substrate, the gasket formed so as to isolate the region formed by the module interconnect and the patch from a surrounding environment, when the module is coupled with the patch.

In aspects, the module interconnect may include an electrically conducting magnetic element, and the patch may include one or more ferromagnetic regions coupled to the substrate, the magnetic elements arranged so as to physically and/or electrically couple the module to the patch when the magnetic elements are aligned with the ferromagnetic regions. In aspects, the ferromagnetic regions may be formed from stretchable pseudo elastic material and/or may be printed onto the substrate. In aspects, the module and/or the patch may include one or more fiducial markings to visually assist with the alignment of the module to the patch during coupling thereof.

According to aspects there is provided a kit for monitoring one or more physiologic, physical, and/or electrophysiological signals from a subject, and/or resource tracking signals for resources associated with a subject, including one or more patches in accordance with the present disclosure, one or more modules in accordance with the present disclosure, a recharging bay in accordance with the present disclosure, and one or more accessories in accordance with the present disclosure. One or more of the accessories may include an adhesive removing agent configured to facilitate substantially pain free removal of one or more of the patches from a subject.

According to aspects there is provided a service system for managing the collection of physiologic and/or resource tracking data from a customer, including a customer data management service, configured to generate and/or store the customer profile referencing customer preferences, data sets, and/or monitoring sessions, an automated product delivery service configured to provide the customer with one or more monitoring products, resources and/or or supplies in accordance with the present disclosure, and a datacenter configured to store, analyze, and/or manage the data obtained from the customer during one or more monitoring sessions.

In aspects, the service system may include a report generating service configured to generate one or more monitoring reports based upon the data obtained during one or more monitoring sessions, a report generating service coupled to the datacenter configured to generate one or more monitoring reports based upon the data obtained during one or more monitoring sessions, and/or a recurrent billing system configured to bill the customer based upon the number or resources consumed, the data stored, and/or the reports generated throughout the course of one or more monitoring sessions.

In aspects, the method may include hot swapping one or more of the devices without interrupting the step of obtaining, and/or calibrating one or more of the devices while on the subject.

In aspects, the method may include determining the position and/or orientation of one or more of the devices on the subject, and/or determining the position and/or orientation from a photograph, a video, or a surveillance video.

In aspects, one or more steps of a method in accordance with the present disclosure may be performed at least in part by one or more devices, patches, modules, and/or systems each in accordance with the present disclosure.

In aspects, one or more of the devices may include an orientation sensor, the orientation sensor configured to obtain an orientation signal, the processor configured to receive the orientation signal or a signal generated therefrom, and to incorporate the orientation signal into the analysis. Some non-limiting examples of orientation sensors include one or more of an altimeter, a barometer, a tilt sensor, a gyroscope, combinations thereof, or the like.

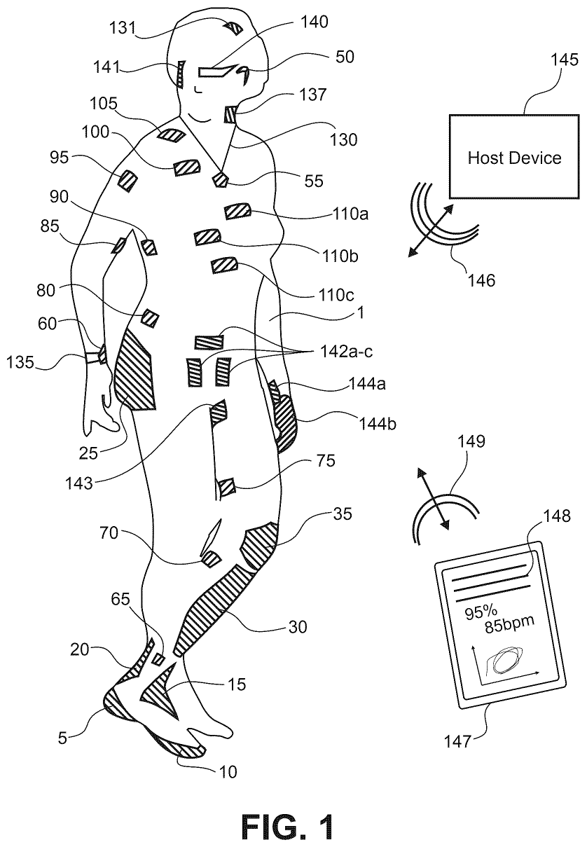

A resource monitoring system, which may include or operate in conjunction with a modular physiologic monitoring system, in some embodiments, includes one or more sensing devices, which may be placed or attached to one or more sites on the subject, on resource supply containers, on equipment that is carried by, used by or otherwise associated with the subject, etc. One or more of the sensing devices may be placed “off” the subject, such as one or more sensors (e.g., cameras, acoustic sensors, etc.) that are not physically attached to the subject. The sensing devices may be utilized to establish whether or not an event is occurring and to determine one or more characteristics of the event by monitoring and measuring physiologic parameters of the subject and/or resource tracking signals or resources used by the subject. The determination of whether an event has occurred or is occurring may be made by a device that is at least partially external and physically distinct from the one or more sensing devices, such as a host device in wired or wireless communication with the sensing devices as described below with respect to FIG. 1. The resource monitoring system may include one or more stimulating devices, which again may be any combination of devices that are attached to the subject or placed “off” the subject, to apply a stimulus to the subject in response to a detected event. Various types of stimulus may be applied, including but not limited to stimulating via thermal input, vibration input, mechanical input, a compression or the like with an electrical input, etc.

The sensing devices of a resource monitoring system, such as patch-module pairs described below with respect to FIG. 1, may be used to track resource usage, as will be described in further detail below. The sensing devices of the resource monitoring system, or a host device configured to receive data or measurements from the sensing devices, may be utilized to monitor for one or more events (e.g., through analysis of signals measured by the sensing devices, from metrics derived from the signals, etc.). Such events may include, but are not limited to, indications that a subject is out of one or more resources, is predicted to be out of or otherwise deplete one or more resources within some designated threshold period of time, indications that a rate of usage of one or more resources will result in the subject being out of such resources before a scheduled resource replenishment, etc. The stimulating devices of the resource monitoring system may be configured to deliver one or more stimuli (e.g., electrical, vibrational, acoustic, visual, etc.) to the subject in response to such events (e.g., to communicate that the subject is out of one or more resources, is predicted to be out of one or more resources within some designated threshold period of time, that a rate of usage of one or more resources will result in the subject being out of such resources before a scheduled resource replenishment, etc.). The stimulating devices may receive a signal from one or more of the sensing devices or a host device, and provide the stimulation in response to the received signal.

FIG. 1 shows aspects of a resource monitoring system (e.g., which may include, be part of, or operate in conjunction with a modular physiologic monitoring system) in accordance with the present disclosure. In FIG. 1, a subject 1 is shown with a number of patches and/or patch-module pairs (or, more generally, “devices” which may be configured for sensing and/or stimulation, where the sensing may include physiologic monitoring and/or resource tracking) each in accordance with the present disclosure attached thereto at sites described below, a host device 145 in accordance with the present disclosure, a feedback/user device 147 in accordance with the present disclosure displaying some data 148 based upon signals obtained from the subject 1, and one or more feedback devices 135, 140, in accordance with the present disclosure configured to convey to the subject 1 one or more aspects of the signals or information gleaned therefrom. In some embodiments, the feedback devices 135, 140 may also or alternatively function as resource sensing devices and/or stimulating devices. The host device 145, the user device 147, the patches and/or patch-module pairs, and/or the feedback devices 135, 140 may be configured for wireless communication 146, 149 during a monitoring session.

In aspects, a patch-module pair may be adapted for placement almost anywhere on the body of a subject 1. As shown in FIG. 1, some sites may include attachment to the cranium or forehead 131, the temple, the ear or behind the ear 50, the neck, the front, side, or back of the neck 137, a shoulder 105, a chest region with minimal muscle mass 100, integrated into a piece of ornamental jewelry 55 (may be a host, a hub, a feedback device, etc.), arrangement on the torso 110a-c, arrangement on the abdomen 80 for monitoring movement or breathing, below the rib cage 90 for monitoring respiration (generally on the right side of the body to substantially reduce EKG influences on the measurements), on a muscle such as a bicep 85, on a wrist 135 or in combination with a wearable computing device 60 on the wrist (e.g., a smart watch, a fitness band, etc.), on a buttocks 25, on a thigh 75, on a calf muscle 70, on a knee 35 particularly for proprioception based studies and impact studies, on a shin 30 primarily for impact studies, on an ankle 65, over an Achilles tendon 20, on the front or top of the foot 15, on a heel 5, or around the bottom of a foot or toes 10. Other sites for placement of such devices are envisioned. Selection of the monitoring and/or stimulating sites is generally determined based upon the intended application of the patch-module pairs described herein.

Additional placement sites on the abdomen, perineal region 142a-c, genitals, urogenital triangle, anal triangle, sacral region, inner thigh 143, or the like may be advantageous in the assessment of autonomic neural function of a subject. Such placements regions may be advantageous for assessment of parasympathetic nervous system (PNS) activity, somatosensory function, assessment of sympathetic nervous system (SNS) functionality, etc.

Placement sites on the wrist 144a, hand 144b or the like may be advantageous for interacting with a subject, such as via performing a stress test, performing a thermal stress test, performing a tactile stress test, monitoring outflow, afferent traffic, efferent traffic, etc.

Placement sites on the nipples, areola, lips, labia, clitoris, penis, the anal sphincter, levator ani muscle, over the ischiocavernous muscle, deep transverse perineal muscle, labium minus, labium majus, one or more nerves near the surface thereof, posterior scrotal nerves, perineal membrane, perineal nerves, superficial transverse perineal nerves, dorsal nerves, inferior rectal nerves, etc., may be advantageous for assessment of autonomic neural ablation procedures, autonomic neural modulation procedures, assessment of the PNS of a subject, assessment of sexual dysfunction of a subject, etc.

Placement sites on the face 141, over ocular muscles, near the eye, over a facial muscle (e.g., a nasalis, temporalis, zygomaticus minor/major, orbicularis oculi, occipitofrontalis), near a nasal canal, over a facial bone (e.g., frontal process, zygomatic bone/surface, zygomaticofacial foreman, malar bone, nasal bone, frontal bone, maxilla, temporal bone, occipital bone, etc.), may be advantageous to assess ocular function, salivary function, sinus function, interaction with the lips, interaction with one or more nerves of the PNS (e.g., interacting with the vagus nerve within, on, and/or near the ear of the subject), etc.

In aspects, a system in accordance with the present disclosure may be configured to monitor one or more physiologic parameters of the subject 1 before, during, and/or after one or more of, a stress test, consumption of a medication, exercise, a rehabilitation session, a massage, driving, a movie, an amusement park ride, sleep, intercourse, a surgical, interventional, or non-invasive procedure, a neural remodeling procedure, a denervation procedure, a sympathectomy, a neural ablation, a peripheral nerve ablation, a radio-surgical procedure, an interventional procedure, a cardiac repair, administration of an analgesic, a combination thereof, or the like. In aspects, a system in accordance with the present disclosure may be configured to monitor one or more aspects of an autonomic neural response to a procedure, confirm completion of the procedure, select candidates for a procedure, follow up on a subject after having received a procedure, assess the durability of a procedure, or the like (e.g., such as wherein the procedure is a renal denervation procedure, a carotid body denervation procedure, a hepatic artery denervation procedure, a LUTs treatment, a bladder denervation procedure, a urethral treatment, a prostate ablation, a prostate nerve denervation procedure, a cancer treatment, a pain block, a neural block, a bronchial denervation procedure, a carotid sinus neuromodulation procedure, implantation of a neuromodulation device, tuning of a neuromodulation device, etc.).

Additional details regarding modular physiologic monitoring systems, kits and methods are further described in PCT application serial no. PCT/US2014/041339, published as WO 2014/197822 and titled “Modular Physiologic Monitoring Systems, Kits, and Methods,” PCT application serial no. PCT/US2015/043123, published as WO 2016/019250 and titled “Modular Physiologic Monitoring Systems, Kits, and Methods,” PCT application serial no. PCT/US2017/030186, published as WO 2017/190049 and titled “Monitoring and Management of Physiologic Parameters of a Subject,” PCT application serial no. PCT/US2018/062539, published as WO 2018/098073 and titled “Continuous Long-Term Monitoring of a Subject,” PCT application serial no. PCT/US2018/043068, published as WO 2019/023055 and titled “Physiologic Monitoring Kits,” PCT application serial no. PCT/2019/033036, published as WO 2019/226506 and titled “Monitoring Physiologic Parameters for Timing Feedback to Enhance Performance of a Subject During an Activity,” PCT application serial no. PCT/US2020/031851, published as WO 2020/227514 and titled “Monitoring and Processing Physiological Signals to Detect and Predict Dysfunction of an Anatomical Feature of an Individual,” PCT application serial no. PCT/US2021033441, published as WO 2021/236948 and titled “Gateway Device Facilitating Collection and Management of Data from a Body Area Network to Study Coordinating System,” PCT application serial no. PCT/US2021/028611, published as WO 2021/216847 and titled “Visualizing Physiologic Data Obtained from Subjects,” PCT application serial no. PCT/US2021/033442, published as WO 2021/236949 and titled “Non-Invasive Detection of Anomalous Physiologic Events Indicative of Hypovolemic Shock of a Subject,” PCT application serial no. PCT/US2021/041414, published as WO 2022/015719 and titled “Wearable Sensor System Configured for Monitoring and Modeling Health Data,” PCT application serial no. PCT/US2021041418, published as WO 2022/015722 and titled “Wearable Sensor System Configured for Facilitating Telemedicine Management,” and PCT application serial no. PCT/US2021/041420, published as WO 2022/015724 and titled “Wearable Sensor System Configured for Alerting First Responders and Local Caregivers,” the disclosures of which are incorporated by reference herein in their entirety.

In some embodiments, resource monitoring systems may include sensing and stimulating devices that are physically distinct, such as sensing and stimulating devices that are physically attached to a subject at varying locations. For example, the sensing and stimulating devices may include different ones of the patch-module pairs described above with respect to FIG. 1. In other embodiments, one or more devices may provide both monitoring and stimulating functionality. For example, one or more of the patch-module pairs described above with respect to FIG. 1 may be configured to function as both a sensing device and a stimulating device. It is to be appreciated, however, that embodiments are not limited solely for use with the patch-module pairs of FIG. 1 as sensing and stimulating devices. Various other types of sensing and stimulating devices may be utilized, including but not limited to sensors that are “off-body” with respect to subject 1.

The sensing and/or stimulating devices of a resource monitoring system may be configured for radio frequency (RF) or other wireless and/or wired connection with one another and/or a host device. Such RF or other connection may be used to transmit or receive feedback parameters or other signaling between the sensing and stimulating devices. The feedback, for example, may be provided based on measurements of physiologic parameters and/or resource tracking signals that are obtained using the sensing devices to determine when events are occurring. Various thresholds for stimulation that are applied by the stimulating devices may, in some embodiments, be determined based on such feedback. Thresholds may relate to the amplitude or frequency of electric or other stimulation. Thresholds may also be related to whether to initiate stimulation by the stimulating devices based on the feedback.

During and/or after stimulus is applied with the stimulating devices, the sensing devices may monitor the physiologic response of the subject. If stimulation is successful in achieving a desired response, the stimulation may be discontinued. Otherwise, the type, timing, etc., of stimulation may be adjusted.

In some embodiments, a user of the resource monitoring system may set preferences for the stimulus type, level, and/or otherwise personalize the sensation during a setup period or at any point during use of the resource monitoring system. The user of the resource monitoring system may be the subject being monitored and stimulated by the sensing devices and stimulating devices, or a doctor, nurse, physical therapist, medical assistant, caregiver, support staff or personal, etc., of the subject being monitored and stimulated. The user may also have the option to disconnect or shut down the resource monitoring system at any time, such as via operation of a switch, pressure sensation, voice operated instruction, etc.

Stimulus or feedback which may be provided via one or more stimulating devices in a resource monitoring system may be in various forms, including physical stimulus (e.g., electrical, thermal, vibrational, pressure, stroking, a combination thereof, or the like), optical stimulus, acoustic stimulus, etc.

Physical stimulus may be provided in the form of negative feedback, such as in a brief electric shock or impulse as described above. Data or knowledge from waveforms applied in conducted electrical weapons (CEWs), such as in electroshock devices, may be utilized to avoid painful stimulus. Physical stimulus may also be provided in the form of positive feedback, such as in evoking pleasurable sensations by combining non-painful electrical stimulus with pleasant sounds, music, lighting, smells, etc. Physical stimulus is not limited solely to electrical shock or impulses. In other embodiments, physical stimulus may be provided by adjusting temperature or other stimuli, such as in providing a burst of cool or warm air, a burst of mist, vibration, tension, stretch, pressure, etc.

Feedback provided via physical stimulus as well as other stimulus described herein may be synchronized with, initiated by or otherwise coordinated or controlled in conjunction with one or more monitoring devices (e.g., a host device, one or more sensing devices, etc.). The monitoring devices may be connected to the stimulating devices physically (e.g., via one or more wires or other connectors), wirelessly (e.g., via radio or other wireless communication), etc. Physical stimulus may be applied to various regions of a subject, including but not limited to the wrist, soles of the feet, palms of the hands, nipples, forehead, ear, mastoid region, the skin of the subject, etc.

Optical stimulus may be provided via one or more stimulating devices. The optical stimulus may be positive or negative (e.g., by providing pleasant or unpleasant lighting or other visuals). Acoustic stimulus similarly may be provided via one or more stimulating devices, as positive or negative feedback (e.g., by providing pleasant or unpleasant sounds). Acoustic stimulus may take the form of spoken words, music, etc. Acoustic stimulus, in some embodiments may be provided via smart speakers or other electronic devices such as Amazon Echo®, Google Home®, Apple Home Pod®, etc. The stimulus itself may be provided so as to elicit a particular psychophysical or psychoacoustic effect in the subject, such as directing the subject to stop an action, to restart an action, to adjust an action, etc.

In some embodiments, a resource monitoring system may be configured to provide multi-modal stimuli to a subject. Multi-modal approaches use one or more forms of stimulation (e.g., thermal and electrical, mechanical and electrical, etc.) in order to mimic another stimulus to trick local nerves into responding in the same manner to the mimicked stimulus. In addition, in some embodiments multi-modal stimulus or input may be used to enhance a particular stimulus. For example, adding a mimicked electrical stimulus may enhance the effect of a thermal stimulus.

Resource monitoring systems may use pulses across space and time (e.g., frequency, pulse trains, relative amplitudes, etc.) to mimic vibration, comfort or discomfort, mild or greater pain, wet sensation, heat/cold, training neuroplasticity, taste (e.g., using a stimulating device placed in the mouth or on the tongue of a subject to mimic sour, sweet, salt, bitter or umami flavor), tension or stretching, sound or acoustics, sharp or dull pressure, light polarization (e.g., linear versus polar, the “Haidinger Brush”), light color or brightness, etc.

Stimulus amplification may also be provided by a resource monitoring system using multi-modal input. Stimulus amplification represents a hybrid approach, wherein a first type of stimulus may be applied and a second, different type of stimulus provided to enhance the effect of the first type of stimulus. As an example, a first stimulus may be provided via a heating element, where the heating element is augmented by nearby electrodes or other stimulating devices that amplify and augment the heating stimulus using electrical mimicry in a pacing pattern. Electrical stimulus may also be used as a supplement or to mimic various other types of stimulus, including but not limited to vibration, heat, cold, etc. Different, possibly unique, stimulation patterns may be applied to the subject, with the central nervous system and peripheral nervous system interpreting such different or unique stimulation patterns as different stimulus modalities.

Another example of stimulus augmentation is sensing a “real” stimulus, measuring the stimulus, and constructing a proportional response by mimicry such as using electric pulsation. The real stimulus, such as sensing heat or cold from a Peltier device, may be measured by electrical-thermal conversion. This real stimulus may then be amplified using virtual mimicry, which may provide energy savings and the possibility of modifying virtual stimulus to modify the perception of the real stimulus.

In some embodiments, the stimulating devices in a resource monitoring system include an electrode array that attaches (e.g., via an adhesive or which is otherwise held in place) to a preferred body part. One or more of the stimulating devices may include a multiplicity of both sensing and stimulation electrodes, including different types of sensing and/or stimulation electrodes. The sensing electrodes on the stimulation devices, in some embodiments, may be distinct from sensing devices in the resource monitoring system used to measure resource tracking signals.

A test stimulus may be initiated in a pattern in the electrode array, starting from application via one or a few of the stimulation electrodes and increasing in number over time to cover an entire or larger portion of the electrode array. The test stimulus may be used to determine the subject's response to the applied stimulation. Sensing electrodes on the stimulation devices may be used to monitor the application of the stimulus. The electrode array may also be used to record a desired output. As such, one or more of the electrodes in the array may be configured so as to measure the local evoked response associated with the stimulus itself. Such an approach may be advantageous to confirm capture of the target nerves during use. By monitoring the neural response to the stimulus, the stimulus parameters including amplitude, duration, pulse number, etc., may be adjusted while ensuring that the target nerves are enlisted by the stimulus in use.

The test stimulus may migrate or be applied in a pattern to different electrodes at different locations in the electrode array. The response to the stimulus may be recorded or otherwise measured, using sensing devices in a modular physiologic monitoring system and/or one or more of the sensing electrodes of the stimulating devices. The response to the test stimulus may be recorded or analyzed to determine an optimal sensing or application site for the stimulus to achieve a desired effect or response in the subject. Thus, the test stimulus may be utilized to find an optimal sensing (e.g., dermatome driver) location. This allows for powerful localization for optimal pacing or other application of stimulus, which may be individualized for different subjects.

A stimulating device applied to the subject via an adhesive (e.g., an adhesively applied stimulating device), may be in the form of a disposable or reusable unit, such as a patch and or patch-module or patch/hub pair as described above with respect to FIG. 1. An adhesively applied stimulating device, in some embodiments, includes a disposable interface configured so as to be thin, stretchable, able to conform to the skin of the subject, and sufficiently soft for comfortable wear. The disposable interface may be built from very thin, stretchable and/or breathable materials, such that the subject generally does not feel the device on his or her body.

Actuation means of the adhesively applied stimulating device may be applied over a small region of the applied area of the subject, such that the adhesive interface provides the biasing force necessary to counter the actuation of the actuation means against the skin of the subject.

Adhesively applied stimulating devices may be provided as two components-a disposable body interface and a reusable component. The disposable body interface may be applied so as to conform to the desired anatomy of the subject, and wrap around the body such that the reusable component may interface with the disposable component in a region that is open and free from a natural interface between the subject and another surface.

An adhesively applied stimulating device may also be a single component, rather than a two component or other multi-component arrangement. Such a device implemented as a single component may include an adhesive interface to the subject including two or more electrodes that are applied to the subject. Adhesively applied stimulating devices embodied as a single component provide potential advantages such as easier application to the body of the subject, but may come at a disadvantage with regards to one or more of breathability, conformity, access to challenging interfaces, etc., relative to two component or multi-component arrangements.

A non-contacting stimulating device may be, for example an audio and/or visual system, a heating or cooling system, etc. Smart speakers and smart televisions or other displays are examples of audio and/or visual non-contacting stimulation devices. A smart speaker, for example, may be used to provide audible stimulus to the subject in the form of an alert, a suggestion, a command, music, other sounds, etc. Other examples of non-contacting stimulating devices include means for controlling temperature such as fans, air conditioners, heaters, etc.

One or more stimulating devices may also be incorporated in other systems, such as stimulating devices integrated into equipment that a subject interfaces with. Such equipment may include, for example, clothing or other gear that can provide a stimulus to the subject in response to a command, feedback signal or control signal generated based on measurement of resource tracking signals obtained utilizing one or more sensing devices.

Although the disclosure has discussed devices attached to the body for monitoring resource usage by a subject, as well as providing a stimulus, therapeutic stimulus, etc., alternative devices may be considered. Non-contacting devices may be used to obtain movement information, audible information, and the like. Such non-contacting devices may be used in place of or to supplement an on-body system for monitoring resource usage, for applying stimulus, etc. Information captured by non-contacting devices may, on its own or in combination with information gathered from sensing devices on the body, be used to direct the application of stimulus to the subject, via one or more stimulating devices on the body and/or via one or more non-contacting stimulating devices.

In some embodiments, aspects of monitoring resource usage by the subject may utilize sensing devices that are affixed to or embodied within resource supply containers, equipment carried by or otherwise associated with the subject.

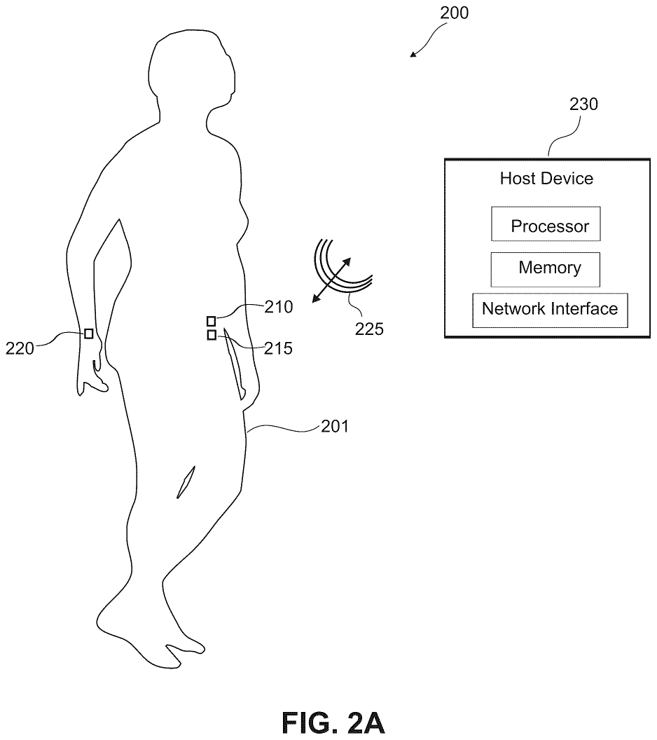

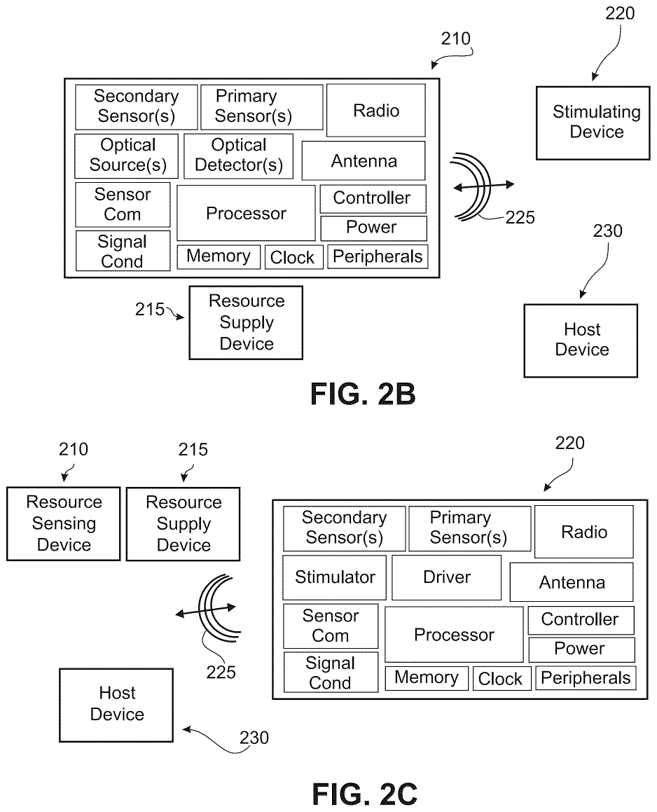

FIGS. 2A-2C show a resource monitoring system 200. The resource monitoring system 200 includes a sensing device 210, a resource supply device 215, and a stimulating device 220 attached to a subject 201 that are in wireless communication 225 with a host device 230. The host device 230 includes a processor, a memory and a network interface.

The processor may comprise a microprocessor, a microcontroller, an application-specific integrated circuit (ASIC), a field-programmable gate array (FPGA) or other type of processing circuitry, as well as portions or combinations of such circuitry elements.

The memory may comprise random access memory (RAM), read-only memory (ROM) or other types of memory, in any combination. The memory and other memories disclosed herein may be viewed as examples of what are more generally referred to as “processor-readable storage media” storing executable computer program code or other types of software programs. Articles of manufacture comprising such processor-readable storage media are considered embodiments of the invention. A given such article of manufacture may comprise, for example, a storage device such as a storage disk, a storage array or an integrated circuit containing memory. The processor may load the computer program code from the memory and execute the code to provide the functionalities of the host device 230.

The network interface provides circuitry enabling wireless communication between the host device 230, the sensing device 210, the resource supply device 215 and the stimulating device 220.

FIG. 2A illustrates a resource monitoring system 200 that includes only a single instance of the sensing device 210, the resource supply device 215 and the stimulating device 220 for clarity. It is to be appreciated, however, that the resource monitoring system 200 may include multiple sensing devices, resource supply devices and/or stimulating devices. In addition, although FIG. 2A illustrates a resource monitoring system 200 in which the sensing device 210, the resource supply device 215 and the stimulating device 220 are attached to the subject 201, embodiments are not limited to such arrangements. As described above, one or more sensing and/or stimulating devices may be part of contacting surfaces or non-contacting devices. Similarly, a resource supply device 215 may not be physically attached to the subject 201. For example, the resource supply device 215 may be associated with a piece of equipment (e.g., a weapon, a medical device, a communications device, etc.) used by the subject 201 and which is in close proximity to the subject 201.

In the embodiment of FIG. 2A, the sensing device 210 is placed in close proximity to the resource supply device 215 near the waist or hip of the subject 201, while the stimulating device 220 is placed on a wrist of the subject 201. The placement of the sensing device 210, the resource supply device 215 and the stimulating device 220 on the subject 201, however, may vary as desired. Also, the host device 230 may be worn by the subject 201, such as being incorporated into a smartwatch or other wearable computing device. The functionality provided by host device 230 may also be provided, in some embodiments, by one or more of the sensing device 210, the resource supply device 215 and the stimulating device 220. In some embodiments, as will be described in further detail below, the functionality of the host device 230 may be provided at least in part using cloud computing resources.

FIG. 2B shows a schematic diagram of aspects of the sensing device 210 in the resource monitoring system 200. The sensing device 210 includes one or more of a processor, a memory device, a controller, a power supply, a power management and/or energy harvesting circuit, one or more peripherals, a clock, an antenna, a radio, a signal conditioning circuit, optical source(s), optical detector(s), a sensor communication circuit, primary sensor(s), and secondary sensor(s). The primary sensors, for example, may be used for resource tracking while the secondary sensors may be used for physiologic monitoring. The sensing device 210 is configured for wireless communication 225 with the stimulating device 220 and the host device 230. The sensing device 210 may be placed in close proximity to, or may in some cases be attached to, affixed to, or integrated with the resource supply device 215.

FIG. 2C shows a schematic diagram of aspects of the stimulating device 220 in the resource monitoring system 200. The stimulating device 220 includes one or more of a processor, a memory device, a controller, a power supply, a power management and/or energy harvesting circuit, one or more peripherals, a clock, an antenna, a radio, a signal conditioning circuit, a driver, a stimulator, primary sensor(s), secondary sensor(s), and a sensor communication circuit. The primary sensors may be used for monitoring application of stimulus by the stimulator, while the secondary sensors may be used for physiologic monitoring and/or resource tracking. The stimulating device 220 is configured for wireless communication 225 with the sensing device 210 and the host device 230.

Communication of data from sensing devices and/or stimulating devices (e.g., patches and/or patch-module pairs) may be performed via a local personal communication device (PCD). Such communication in some embodiments takes place in two parts: (1) local communication between a patch and/or patch-module pair (e.g., via a hub or module of a patch-module pair) and the PCD; and (2) remote communication from the PCD to a back-end server, which may be part of a cloud computing platform and implemented using one or more virtual machines (VMs) and/or software containers. The PCD and back-end server may collectively provide functionality of the host device as described elsewhere herein.

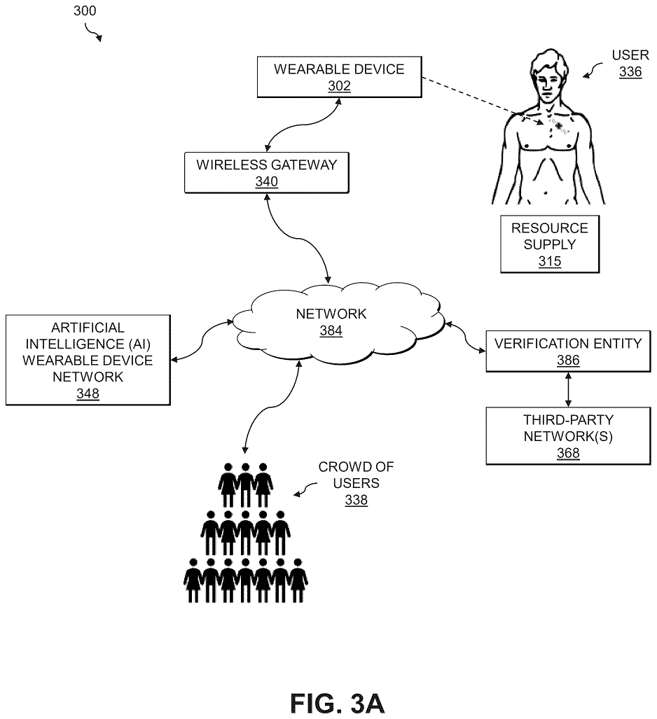

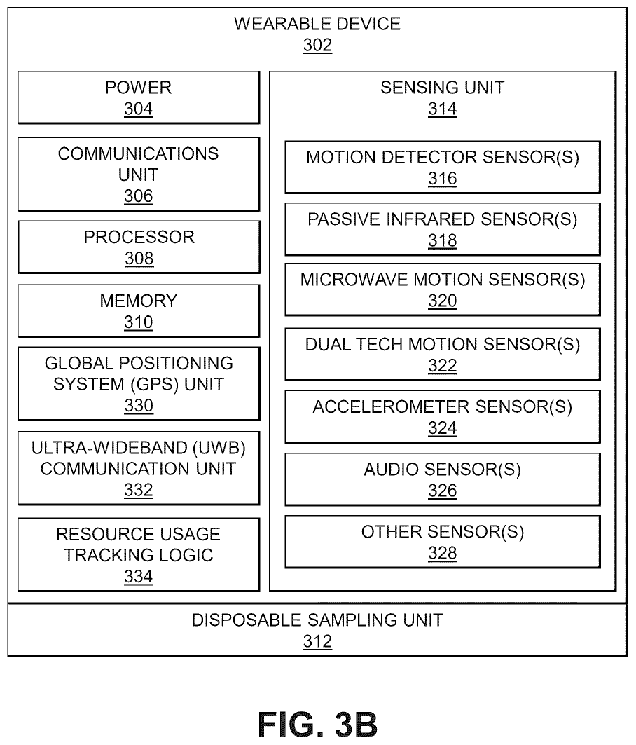

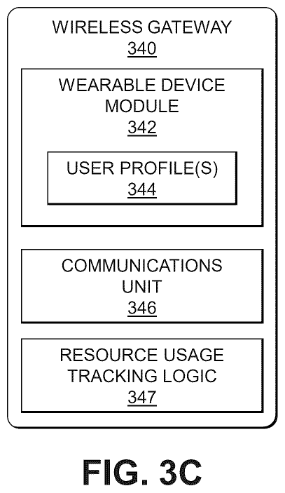

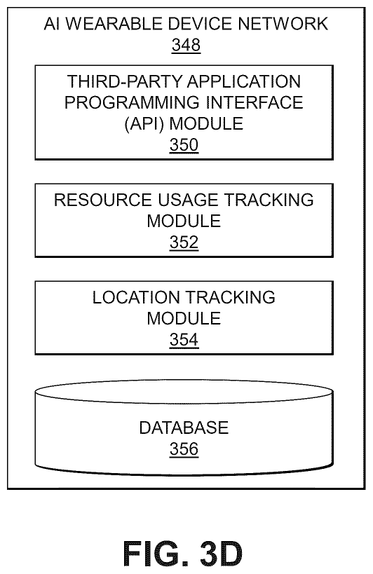

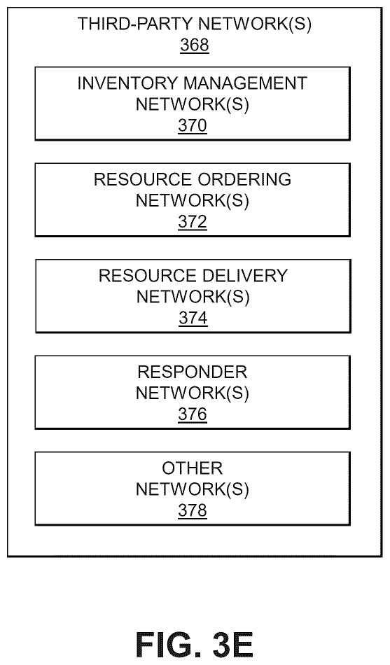

FIGS. 3A-3E show a wearable sensor system 300 configured for monitoring data (e.g., physiologic data, location data, resource usage data, etc.) for a plurality of users, and for analyzing such data. The wearable sensor system 300 provides the capability for assessing and tracking resource usage by a plurality of users (e.g., including user 336 and a crowd of users 338). As shown in FIG. 3A, the wearable sensor system 300 includes a wearable device 302 that is affixed to user 336, the user being associated with a resource supply 315 of one or more resources. Data collected from the user 336 via the wearable device 302 is communicated using a wireless gateway 340 to an artificial intelligence (AI) wearable device network 348 over or via network 384. The network 384 may comprise a physical connection (wired or wireless), the Internet, a cloud communication network, etc. Examples of wireless communication networks that may be utilized include networks that utilize Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. Also coupled to the network 384 is a crowd of users 338 and a verification entity 386 coupled to a set of third-party networks 368. Detailed views of the wearable device 302, wireless gateway 340, AI wearable device network 348 and third-party networks 368 are shown in FIGS. 3B-3E, respectively.

In some embodiments, the wearable device 302 is implemented using one or more patch-module pairs as described above with respect to FIGS. 1 and 2A-2C. The patch-module pairs described above with respect to FIGS. 1 and 2A-2C, however, are just one example of wearable technology that may be used to provide the wearable device 302. Various other types of wearable technology may be used to provide the wearable device in other embodiments, including but not limited to wearables, fashion technology, tech togs and other types of fashion electronics that include “smart” electronic devices (e.g., electronic devices with micro-controllers) that can be incorporated into clothing or worn on the body as implants or accessories. Wearable devices such as activity trackers are examples of Internet of Things (IoT) devices, and such “things” include electronics, software, sensors and connectivity units that are effectors enabling objects to exchange data (including data quality) through the Internet with a manufacturer, operator and/or other connected devices without requiring human intervention. Wearable technology has a variety of applications, which grows as the field itself expands. Wearable technology appears prominently in consumer electronics with the popularization of smartwatches and activity trackers. Apart from commercial uses, wearable technology is being incorporated into navigation systems, advanced textiles, health care, military applications (e.g., monitoring and tracking of soldiers in military and paramilitary environments).

In some embodiments, the wearable device 302 is capable of detecting and collecting resource usage data by the wearer (e.g., user 336). The wearable device 302 can remotely collect and transmit real-time resource usage data to support staff, health care providers and other caretakers responsible for managing users performing activities in different environments. The wearable sensor system 300, in some embodiments, is user-friendly, hypoallergenic, unobtrusive, and cost-effective. In service of enabling remote evaluation of individual resource status, the wearable sensor system 300 is configured to transmit data directly into existing inventory and other resource tracking management systems from remote locations where users are performing different activities. The wearable device 302 is designed to monitor resource usage by a subject (e.g., user 336) over time in different settings or environments. Onboard sensors of the wearable device 302 can quantitatively detect and track resource usage and potentially other information such as physiologic data (e.g., severity of a variety of disease symptoms including fever, coughing, sneezing, vomiting, infirmity, tremor, and dizziness, as well as signs of decreased physical performance and changes in respiratory rate/depth, etc.).

In some embodiments, the wearable device 302 collects resource usage data from the subject user 336 utilizing a combination of a disposable sampling unit 312 and a reusable sensing unit 314 (FIG. 3B). The patch-module pairs described above with respect to FIGS. 1 and 2A-2C are an example implementation of the disposable sampling unit 312 and reusable sensing unit 314. The disposable sampling unit 312 may be formed from a softer-than-skin patch. The wearable device 302, formed from the combination of the disposable sampling unit 312 and reusable sensing unit 314, is illustratively robust enough for military use, yet extremely thin and lightweight. For example, the disposable sampling unit 312 and reusable sensing unit 314 may collectively weigh less than 0.1 ounce, about the same as a U.S. penny. The wearable device 302 may be adapted for placement almost anywhere on the body of the user 336, or which are attached to or integrated with various equipment that is carried by or otherwise associated with the user 336, such as the various placement sites shown in FIG. 1 and described above.

In addition to the disposable sampling unit 312 and reusable sensing unit 314, the wearable device 302 may include a number of other components as illustrated in FIG. 3B. Such components include a power source 304, a communications unit 306, a processor 308, a memory 310, a GPS unit 330, an UWB communication unit 332, and resource usage tracking logic 334.

The power source or component 304 of the wearable device 302, in some embodiments, includes one or more modules with each module including a power source (e.g., a battery, a rechargeable battery, an energy harvesting transducer, a microcircuit, an energy reservoir, a thermal gradient harvesting transducer, a kinetic energy harvesting transducer, a radio frequency energy harvesting transducer, a fuel cell, a biofuel cell, combinations thereof, etc.).

The communications unit 306 of the wearable device 302 may be embodied as communication circuitry, or any communication hardware that is capable of transmitting an analog or digital signal over one or more wired or wireless interfaces. In some embodiments, the communications unit 306 includes transceivers or other hardware for communications protocols, such as Near Field Communication (NFC), WiFi, Bluetooth, infrared (IR), modem, cellular, ZigBee, a Body Area Network (BAN), and other types of wireless communications. The communications unit 306 may also or alternatively include wired communication hardware, such as one or more universal serial bus (USB) interfaces.

The processor 308 of the wearable device 302 is configured to decode and execute any instructions received from one or more other electronic devices and/or servers. The processor 308 may include any combination of one or more general-purpose processors (e.g., Intel® or Advanced Micro Devices (AMD)® microprocessors), one or more special-purpose processors (e.g., digital signal processors or Xilink® system on chip (SOC) field programmable gate array (FPGA) processors, application-specific integrated circuits (ASICs), etc.), etc. The processor 308 is configured in some embodiments to execute one or more computer-readable program instructions, such as program instructions to carry out any of the functions described herein including but not limited to those of the resource usage tracking logic 334 described below. The processor 308 is illustratively coupled to the memory 310, with the memory 310 storing such computer-readable program instructions.

The memory 310 may include, but is not limited to, fixed hard disk drives, magnetic tape, floppy diskettes, optical disks, compact disc read-only memories (CD-ROMs), magneto-optical disks, semiconductor memories such as read-only memory (ROM), random-access memory (RAM), programmable ROM (PROM), erasable PROM (EPROM), electrically erasable PROM (EEPROM), flash memory, magnetic or optical cards, or other type of media/machine-readable medium suitable for storing electronic instructions. The memory 310 may comprise modules implemented as one or more programs. In some embodiments, a non-transitory processor-readable storage medium has stored therein program code of one or more software programs, wherein the program code when executed by at least one processing device (e.g., the processor 308) causes said at least one processing device to perform one or more aspects of the methods, algorithms and process flows described herein.

The processor 308 and memory 310 are an example of a processing device or controller. The controller may comprise a central processing unit (CPU) for carrying out instructions of one or more computer programs for performing arithmetic, logic, control and input/output (I/O) operations specified by the instructions (e.g., as specified by the resource usage tracking logic 334 as described in further detail below). Such computer programs may be stored in the memory 310. The memory 310 provides electronic circuitry configured to temporarily store data that is utilized by the processor 308. In some embodiments, the memory 310 further provides persistent storage for storing data utilized by the processor 308. Although not explicitly shown, other components of the wearable sensor system 300 (e.g., the wireless gateway 340 (FIG. 3C), the AI wearable device network 348, one or more of the third-party networks 368, the verification entity 386, etc.) may also include one or more processors coupled to one or more memories providing processing devices implementing the functionality of such components.

As noted above, the wearable device 302 illustratively includes the disposable sampling unit 312 which may be embodied as a physical interface to the skin of the user 336, or a physical interface configured for attachment to equipment that is carried by or otherwise associated with the user 336. Patches as described elsewhere herein are examples of a disposable sampling unit 312. Such patches are adapted for attachment to a human or animal body (e.g., attachable to the skin thereof, reversibly attachable, adhesively attachable, with a disposable interface that couples to a reusable module, etc.), or to a surface of a resource supply device or other piece of equipment that is carried by or otherwise associated with the user 336. In some embodiments, the disposable sampling unit 312 is part of a system that is capable of modular design, such that various wearable devices or portions thereof (e.g., reusable sensing unit 314) are compatible with various disposable sampling units with differing capabilities. In some embodiments, the patch or more generally the disposable sampling unit 312 allows sterile contact between the user 336 and other portions of the wearable device 302, such as the reusable sensing unit 314. In such embodiments, the other portions of the wearable device 302 (e.g., which may be embodied as a module as described above with respect to FIGS. 1 and 2A-2C) may be returned, sterilized and reused (e.g., by the same user 336 or another user) while the patch or disposable sampling unit 312 is disposed of. In some embodiments, the patch or other disposable sampling unit 312 is suitable for wearing over a duration of time in which the user 336 is undergoing monitoring. In such embodiments, the patch or disposable sampling unit 312 may be disposed of after the monitoring duration has ended.

The reusable sensing unit 314 includes various sensors, such as one or more motion detector sensors 316 (e.g., one or more image or video capture sensors, one or more ultrasonic motion detectors), one or more passive infrared sensors 318, one or more microwave motion sensors 320, one or more dual tech motion sensors 322 (e.g., configured to detect movement using both passive infrared and microwave sensors), one or more accelerometer sensors 324, one or more audio sensors 326, and one or more other sensors 328 (e.g., physiologic monitoring sensors such as temperature sensors, heart rate sensors, respiration sensors, pulse oximetry sensors, etc.). One or more of the sensors 316-328 may be embodied as electric features, capacitive elements, resistive elements, touch sensitive components, analyte sensing elements, printed electrochemical sensors, light sensitive sensing elements, electrodes (e.g., including but not limited to needle electrodes, ionically conducting electrodes, reference electrodes, etc.), electrical traces and/or interconnects, stretch sensing elements, contact interfaces, conduits, microfluidic channels, antennas, stretch resistant features, stretch vulnerable features (e.g., a feature that changes properties reversibly or irreversibly with stretch), strain sensing elements, photo-emitters, photodiodes, biasing features, bumps, touch sensors, pressure sensing elements, interfacial pressure sensing elements, piezoelectric elements, piezoresistive elements, chemical sensing elements, electrochemical cells, electrochemical sensors, redox reactive sensing electrodes, light sensitive structures, moisture sensitive structures, pressure sensitive structures, magnetic structures, bioadhesives, antennas, transistors, integrated circuits, transceivers, sacrificial structures, water soluble structures, temperature sensitive structures, light sensitive structures, light degrading structures, flexible light emitting elements, piezoresistive elements, moisture sensitive elements, mass transfer altering elements, etc.

The accelerometer sensors 324 are configured to measure acceleration of the user 336. Single and multi-axis models of accelerometers may be used to detect the magnitude and direction of the proper acceleration as a vector quantity, and can be used to sense orientation (e.g., based on the direction of weight changes), coordinate acceleration, vibration, shock, and falling in a resistive medium (e.g., a case where the proper acceleration changes, since it starts at zero then increases). The accelerometer sensors 324 may be embodied as micromachined microelectromechanical systems (MEMS) accelerometers present in portable electronic devices such as the wearable device 302. The accelerometer sensors 324 may also be used for sensing muscle contraction for various activities. The accelerometer sensors 324 may detect such activity by measuring the body or extremity center of mass of the user 336.

The audio sensors 326 are configured to convert sound into electrical signals, and may be embodied as one or more microphones or piezoelectric sensors that use the piezoelectric effect to measure changes in pressure, acceleration, temperature, strain, or force by converting them to an electrical charge. In some embodiments, the audio sensors 326 may include ultrasonic transducer receivers capable of converting ultrasound into electrical signals.

It should be noted that the sensors 316-326 described above are presented by way of example only, and that the sensing unit 314 may utilize various other types of sensors 328 as described elsewhere herein. For example, in some embodiments the other sensors 328 include one or more of weight sensors, motion sensors, humidity sensors, cameras, radiofrequency receivers, thermal imagers, radar devices, lidar devices, ultrasound devices, speakers, etc.

The GPS unit 330 is a component of the wearable device 302 configured to detect global position using GPS, a satellite-based radio navigation system owned by the U.S. government and operated by the U.S. Space Force. GPS is one type of global navigation satellite system (GNSS) that provides geolocation and time information to a GPS receiver anywhere on or near the Earth where there is an unobstructed line of sight to four or more GPS satellites.

The UWB communication unit 332 is a component of the wearable device 302 configured to detect UWB radiofrequencies. UWB is a short-range, wireless communication protocol similar to Bluetooth or WiFi, which uses radio waves at a very high frequency. Notably, UWB also uses a wide spectrum of several gigahertz (GHz). The functioning of a UWB sensor is to provide the ability to continuously scan an entire room and provide spatial awareness data to the wearable device 302, improving the localization of the wearable device 302 particularly in conjunction with use of the GPS unit 330.

The resource usage tracking logic 334 is configured to execute various functionality for tracking usage of resources by the user 336, and for communicating resource tracking information between the wearable device 302 and other devices (e.g., wireless gateway 340, other wearable devices and/or associated wireless gateways for users in the crowd of users 338, the AI wearable device network 348, third-party networks 368, etc.). As described in further detail below, the wearable device 302 and/or its associated wireless gateway 340 may be part of a mobile ad-hoc network (MANET) that leverages multiple radio types and distinct physical layers. Such different radio types and distinct physical layers may utilize the communications unit 306 and UWB communication unit 332, for switching among radio types and standards including but not limited to UWB, NFC, WiFi, Bluetooth, Bluetooth Low Energy (BLE), infrared (IR), modem, cellular (e.g., including but not limited to Long-Term Evolution (LTE), LTE Machine Type Communication (LTE-MTC or LTE-M), etc.), low-power wide-area network (LPWAN) radio technology standards such as Narrowband Internet of Things (NB-IoT) and network modulation techniques such as LoRa, Zigbee, BAN, etc. Software programs or computer instructions for the resource usage tracking logic 334 when executed causes the processor 308 to obtain resource usage tracking data (e.g., from one or more sensing devices associated with the user 336), the resource usage tracking data characterizing availability of one or more resources utilized by the subject. The resource usage tracking logic 334 when executed also causes the processor 308 to determine, based at least in part on the received resource usage tracking data, one or more resource usage metrics for the one or more resources, and to generate, based at least in part on the determined one or more resource usage metrics, one or more feedback signals. The resource usage tracking logic 334 when executed further causes the processor 308 to transmit the generated one or more feedback signals to at least one additional device.

The user 336 may be a human or animal to which the wearable device 302 is attached. Resource usage tracking data collected by the wearable device 302 may be provided to AI wearable device network 348 for analysis, with portions of such analysis being provided to one or more of the third-party networks 368 for various purposes. Communication of the resource usage tracking data from the wearable device 302 to the AI wearable device network 348 may take place via a wireless gateway 340, with the communication between the wireless gateway 340 and the AI wearable device network 348 taking place over one or more networks 384.

As shown in FIG. 3C, the user 336 may configure the wireless gateway 340 to include a user profile 344. The user profile 344 may include various resource usage tracking data about the user 336 that may not be obtained by sensors 316-328 of the wearable device 302. The user profile 344, for example, may include information such as a name (e.g., first, last and middle name), biological sex, age (e.g., in years), weight (e.g., in pounds, kilograms, etc.), and height (e.g., in feet or inches, in meters, etc.). The user profile 344 may also include known diseases and disorders (e.g., asthma, allergies, current medications, family medical history, other medical data, etc.), where such information may include Protected Health Information (PHI) regulated by American Health Insurance Portability and Accountability Act (HIPAA) or other applicable rules and regulations. PHI includes individually identifiable health information that relates to one or more of: the past, present, or future physical or mental health or condition of an individual; provision of health care to the individual by a covered entity (e.g., a hospital or doctor); the past, present, or future payment for the provision of health care to the individual; telephone numbers, fax numbers, email addresses, Social Security numbers, medical record numbers, health plan beneficiary numbers, license plate numbers, uniform resource locators (URLs), full-face photographic images or any other unique identifying numbers, characteristics, codes, or combination thereof that allows identification of an individual. The user profile 344 may further include an emergency contact (e.g., name, phone number, address, etc.), next of kin (e.g., name, phone number, address, etc.), preferred hospital (e.g., name, phone number, address, etc.) and primary care physician (PCP) of the user 336 (e.g., name, phone number, place of business, etc.). The user profile 344 may further include local caregiver information (e.g., name, phone number, address, etc.) and preferred first responder network information (e.g., name, phone number, address, etc.). The local caregiver may be, for example, a nursing agency, a private caregiver such as a family member, a nursing home, or other local caregivers such as physical therapists, chiropractors, pharmacists, pediatricians, acupuncture specialists, massage therapists, etc. In some cases, the local caregiver is associated with one or more telemedicine networks. The preferred first responder network may be, for example, a local hospital and/or a local ambulatory rescue agency. In some embodiments, the preferred first responder network may be an interface with an emergency calling network (e.g., 911). The user profile 344 may further include types of resources consumed by the user 336 that are to be tracked, suppliers for such resources, payment information for such resources, etc.

The wireless gateway 340 sends the resource usage tracking data obtained from the user 336 by the wearable device 302 utilizing communications unit 346, which may comprise any type of transceiver for coupling the wireless gateway 340 to the network 384. The communications unit 346 of the wireless gateway 340 may be embodied as communication circuitry or any communication hardware capable of transmitting an analog or digital signal over wired or wireless network interfaces. Such network interfaces may support not only communication with the AI wearable device network 348 over network 384, but also communications between the wearable device 302 and the wireless gateway 340. Any combination of network types may be utilized, including but not limited to UWB, NFC, WiFi, Bluetooth, BLE, IR, modem, cellular, ZigBee, BAN, etc. The wireless gateway 340 may also be provisioned with resource usage tracking logic 347, which provides functionality similar to that of the resource usage tracking logic 334 but for communicating resource usage tracking data between the wireless gateway 340 and other devices (e.g., wearable device 302, other wearable devices and/or associated wireless gateways for users in the crowd of users 338, the AI wearable device network 348, third-party networks 368, etc.).

The wireless gateway 340 may be, for example, a smartphone, a tablet, a laptop or desktop computer, an Internet-connected modem, a wireless router or standalone wireless hub device connected to the Internet, etc. The wireless gateway 340, in some embodiments, may itself comprise or be incorporated into one or more wearable devices (e.g., a smartwatch, an activity tracker, etc.). In some cases, the wireless gateway 340 may be part of the wearable device 302, or vice versa. The wireless gateway 340 is illustratively a smart device that is owned or controlled by the user 336, such as a smartphone, and allows rapid onboarding of wearable devices such as wearable device 302 to the AI wearable device network 348.

The wireless gateway 340 includes a wearable device module 342 that provides software programs or computer instructions for providing functionality of the wireless gateway 340. Although not shown in FIG. 3C, the wireless gateway 340 is assumed to comprise at least one processing device or controller including a processor coupled to a memory for executing the functionality of the wearable device module 342. Such functionality includes receiving resource usage tracking data (possibly along with physiologic sensor data and localization data) from the wearable device 302 via the communications unit 346, and possibly performing a preliminary analysis of the received data. Such analysis may be based at least in part on information stored in the user profile 344. Based on such analysis, the wearable device module 342 may determine whether any immediate notifications should be provided to the user 336. Such notifications may comprise, for example, indications that the user 336 is out of or is predicted to be out of one or more types of resources within some designated threshold period of time, that a rate of usage of one or more types of resources by the user 336 has changed, etc. In other embodiments, the wearable device 302 functions as a pass-through entity and does not perform such preliminary analysis. Instead, the wireless gateway 340 may provide the data received from the wearable device 302, along with the associated user profile 344, to the AI wearable device network 348 over network 384 as a pass-through entity.

Regardless of whether or not the wireless gateway 340 performs such preliminary analysis, the wearable device module 342 of the wireless gateway 340 may receive any combination of resource usage tracking and ordering information, resource usage tracking data analysis, sensor data analysis, localization analysis, analysis created from a fusion of data from a plurality of sensors from the AI wearable device network 348, etc. At least a portion of the received information is based on analysis of the resource usage tracking data, the sensor data, and/or the localization data and the user profile 344 or information derived therefrom previously provided by the wireless gateway 340 to the AI wearable device network 348. At least a portion of the received information is used to generate notifications or other output via a graphical user interface (GUI) of the wireless gateway 340, the wearable device 302 or another type of local or remote indicator device.

The wearable device module 342 may provide functionality for determining notification settings associated with the user 336, and to execute or deliver notifications in accordance with the determined notification settings. The notification settings, in some embodiments, may specify the types of indicator devices that are part of or otherwise accessible to the wearable device 302 for delivering notifications to the user 336 (or to a doctor, nurse, physical therapist, medical assistant, caregiver, etc. associated with the user 336). The indicator devices in some embodiments may be configured to deliver visual or audible alarms. In other embodiments, the indicator devices may be configured to provide stimulus or feedback via stimulating devices as described elsewhere herein. Such stimulus or feedback, as detailed above, may include physical stimulus (e.g., electrical, thermal, vibrational, pressure, stroking, a combination thereof, or the like), optical stimulus, acoustic stimulus, etc. In some embodiments, notifications may be delivered to remote terminals or devices other than the wearable device 302 associated with user 336. For example, notifications may be delivered to one or more devices associated with a doctor, nurse, physical therapist, medical assistant, caregiver, etc. associated with the user 336.

The notification delivery method may also or alternatively comprise a visual or audible read-out or alert from a “local” device that is in communication with the wearable device 302. The local device may comprise, for example, a mobile computing device such as a smartphone, tablet, laptop etc., or another computing device, that is associated with the user 336. The wearable device 302 is one example of a local device. A local device may also include devices connected to the wearable device 302 via a BAN or other type of local or short-range wireless network (e.g., a Bluetooth network connection).

The notification delivery method may further or alternatively comprise a visual or audible read-out or alert from a “remote” device that is in communication with the wearable device 302 or the wireless gateway 340 via network 384. The remote device may be a mobile computing device such as a smartphone, tablet, laptop, etc., or another computing device (e.g., a telemetry center or unit within a hospital or other facility), that is associated with a doctor, nurse, physical therapist, medical assistant, caregiver, etc. monitoring the user 336. It should be understood that the term “remote” in this context does not necessarily indicate any particular physical distance from the user 336. For example, a remote device to which notifications are delivered may be in the same room as the user 336. The term “remote” in this context is instead used to distinguish from “local” devices (e.g., in that a “local” device in some embodiments is assumed to be owned by, under the control of, or otherwise associated with the user 336, while a “remote” device is assumed to be owned by, under the control of, or otherwise associated with a user or users other than the user 336 such as a doctor, nurse, physical therapist, medical assistance, caregiver, etc.).

The indicator devices may include various types of devices for delivering notifications to the user 336 (or to a doctor, nurse, physical therapist, medical assistant, caregiver, etc. associated with the user 336). In some embodiments, one or more of the indicator devices comprise one or more light emitting diodes (LEDs), a liquid crystal display (LCD), a buzzer, a speaker, a bell, etc., for delivering one or more visible or audible notifications. More generally, the indicator devices may include any type of stimulating device as described herein which may be used to deliver notifications to the user 336 (or to a doctor, nurse, physical therapist, medical assistant, caregiver, etc. associated with the user 336).

FIG. 3A also shows the crowd of users 338, each of which is assumed to provide resource usage tracking data, sensor data and/or localization data obtained by a plurality of wearable devices to the AI wearable device network 348, possibly via respective wireless gateways. The wearable devices and wireless gateways for the crowd of users 338 may be configured in a manner similar to that described herein with respect to the wearable device 302 and wireless gateway 340 associated with the user 336.

The AI wearable device network 348 is configured to receive data (e.g., resource usage tracking data, sensor data, localization data, user profiles, preliminary analysis of sensor and localization data, etc.) from the wireless gateway 340 and the crowd of users 338. The AI wearable device network 348 analyzes the received data using various software modules implementing AI algorithms for determining resource usage (e.g., including resource usage trends), occurrence of events, event classification, etc. As shown in FIG. 3D, such modules include a third-party application programming interface (API) module 350, a resource usage tracking module 352, and a location tracking module 354. The AI wearable device network 348 also includes a database 356 configured to store the received data, results of analysis on the received data, data obtained from third-party networks 368, etc.

In some embodiments, the AI wearable device network 348 is implemented as an application or applications running on one or more physical or virtual computing resources. Physical computing resources include, but are not limited to, smartphones, laptops, tablets, desktops, wearable computing devices, servers, etc. Virtual computing resources include, but are not limited to, VMs, software containers, etc. The physical and/or virtual computing resources implementing the AI wearable device network 348, or portions thereof, may be part of a cloud computing platform. A cloud computing platform includes one or more clouds providing a scalable network of computing resources (e.g., including one or more servers and databases). In some embodiments, the clouds of the cloud computing platform implementing the AI wearable device network 348 are accessible via the Internet over network 384. In other embodiments, the clouds of the cloud computing platform implementing the AI wearable device network 348 may be private clouds where access is restricted (e.g., such as to one or more credentialed medical professionals or other authorized users). In these and other embodiments, the AI wearable device network 348 may be considered as forming part of a resource usage tracking network comprising at least one server and at least one database (e.g., the database 356) storing resource usage tracking data pertaining to a plurality of users (e.g., the user 336 and crowd of users 338).

The database 356 provides a data store for information about resource usage by the user 336 and the crowd of users 338 (e.g., historical resource usage, historical resource deployment to the user 336 and the crowd of users 338, etc.). Although shown as being implemented internal to the AI wearable device network 348 in FIG. 3D, it should be appreciated that the database 356 may also be implemented at least in part external to the AI wearable device network 348 (e.g., as a standalone server or storage system). The database 356 may be implemented as part of the same cloud computing platform that implements the AI wearable device network 348.

The AI wearable device network 348 may exchange various information with one or more third-party networks 368. As shown in FIG. 3E, the third-party networks 368 may include any combination of one or more inventory management networks 370, one or more resource ordering networks 372, one or more resource delivery networks 374, one or more responder networks 376, and one or more other networks 378.

Under certain circumstances, as permitted by the verification entity 386, one or more of the third-party networks 368 may receive data and analysis from the AI wearable device network 348, for various purposes including but not limited to tracking inventory of supplies or other resources provisioned to the user 336 and/or the crowd of users 338 (e.g., using the inventory management networks 372), ordering supplies or other resources to replenish the user 336 and/or the crowd of users 338 (e.g., using the resource ordering networks 372), delivering supplies or other resources to the user 336 and/or the crowd of users 338, including transferring resources between the user 336 and/or the crowd of users 338 (e.g., using the resource delivery networks 374), deploying personnel or resources to assist the user 336 and/or the crowd of users 338 (e.g., using the responder networks 376). The responder networks 376, for example, may be associated with an entity that oversees operations performed by the user 336 and/or the crowd of users 338. In a military setting, the responder networks 376 may be associated with a military and/or paramilitary entity that oversees operations performed by the user 336 and/or the crowd of users 338. For example, the responder networks 376 may be associated with an entity having an ability to deliver equipment and/or resources to the users 336 and/or the crowd of users 338 (e.g., possibly operating in conjunction with other ones of the third-part networks 368, such as the resource ordering networks 372 and resource delivery networks 374). Consider, as an example, a military environment in which the responder networks 376 may utilize drones and/or robotics equipment capable of delivering ammunition or other supplies to the subject 336 and/or the crowd of users 338.

The exchange of information between the AI wearable device network 348 and third-party networks 368 may involve use of a verification entity 386, which ensures data security in accordance with applicable rules and regulations. The AI wearable device network 348 utilizes the third-party API module 350 to perform such verification of the third-party networks 368 utilizing the verification entity 386, before providing any data or analysis thereof related to the user 336 or crowd of users 338 to any of the third-party networks 368. It should be noted that, if desired, any data or analysis related to the user 336 or crowd of users 338 may be anonymized prior to being sent to one or more of the third-party networks 368, such as in accordance with privacy settings in user profiles (e.g., user profile 344 associated with the user 336, user profiles associated with respective users in the crowd of users 338, etc.).

The location tracking module 356 is configured to track the location of user 336 and the crowd of users 338, which may be useful for determining availability of resources which can be re-supplied among the user 336 and the crowd of users 338. For example, the resource usage tracking module 352 may determined that the user 336 has run out or is predicted to run out of a particular type of resource within some designated threshold period of time (e.g., before a scheduled replenishment of that particular type of resource), but that one or more users in the crowd of users 338 have excess amounts of that particular type of resource (e.g., that such users are predicted to have at least a designated threshold amount of the particular type of resource remaining at a scheduled replenishment of that particular type of resource). In such cases, the location tracking module 354 may be used to determine if the user 336 is in close proximity to such other users in the crowd of users 338, such that the other users in the crowd of users 338 can transfer some resources of the particular type to replenish the user 336. This may include predictive location tracking by the location tracking module 354, and possibly diverting the user 336 and/or one or more of the other users in the crowd of users 338 such that they will be in close proximity to facilitate such an exchange of resources.

As a specific example, consider the tracking of ammunition by soldiers in a military setting. If a particular solider (e.g., user 336) is determined to be out of a particular type of ammunition using the resource usage tracking module 352, other soldiers (e.g., users in the crowd of users 338) in a same or different unit that carry the same type of ammunition may be analyzed to track whether any has an “excess” amount of the needed ammunition. If so, the location tracking module 354 can determine whether the ammunition may be exchanged or shared among the soldiers such that no individual soldier will run out of the needed ammunition before the end of a mission or deployment (e.g., before an ammunition resupply event).

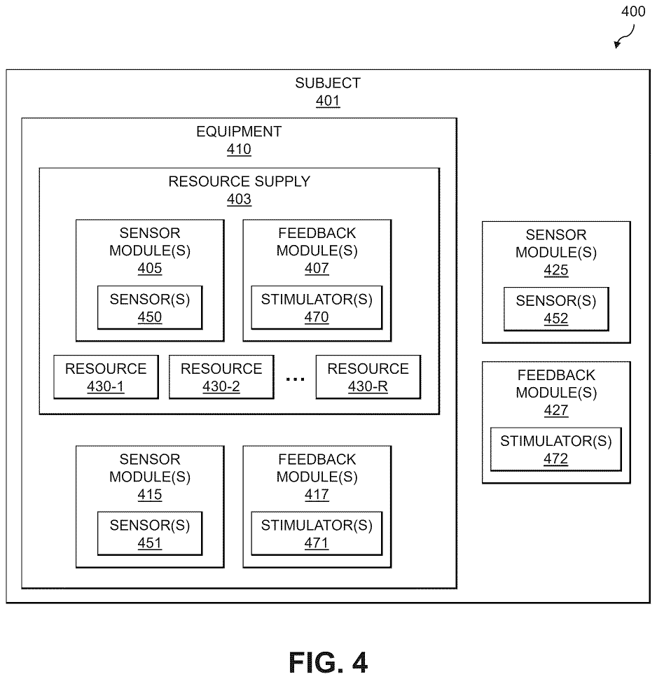

FIG. 4 shows aspects of a resource monitoring system 400. In FIG. 4, a subject 401 has equipment 410 including a resource supply 403 associated with resources 430-1, 430-3, . . . 430-R (collectively, resources 430). The resources 430 may comprise different types of resources that are used by the equipment 410 of the subject 401. In the FIG. 4 example, the resource supply 403 is associated with one or more sensor modules 405 each having one or more sensors 450 and one or more feedback modules 407 each having one or more stimulators 470. Here, the equipment 410 may also be associated with one or more sensor modules 415 having one or more sensors 451 and one or more feedback modules 417 having one or more stimulators 471, where the sensor modules 415 and the feedback modules 417 are “outside” of the resource supply 403. This is in contrast with the sensor modules 405 and feedback modules 407 which are assumed to be attached to or part of the resource supply 403. The subject 401 may be further associated with one or more sensor modules 425 each having one or more sensors 452 and one or more feedback modules 427 each having one or more stimulators 472. Here, the sensor modules 425 and the feedback modules 427 are assumed to be “outside” of the equipment 410 altogether, such as being attached to or worn by the subject 401. The sensor modules 405, 415 and 425 are examples of what are more generally referred to herein as sensing devices, while the feedback modules 407, 417 and 427 are examples of what are more generally referred to herein as stimulating devices.

It should be noted that the feedback modules 407, 417 and 427 are optional. In some embodiments, for example, the feedback modules 427 are present only on the subject 401, and the feedback modules 407 and 417 are omitted. Similarly, sensor modules need not be present on the resource supply 403, the equipment 410 and the subject 401. In some embodiments, for example, the sensor modules 405 are only present on the resource supply 403, and the sensor modules 415 and 425 are omitted. It should also be appreciated that in some embodiments sensor and feedback modules may be combined (e.g., a combined module may include both sensors and stimulators). Various other combinations are possible.

The feedback modules 407, 417 and 427 are configured to convey to the subject 401 one or more aspects of resource tracking signals (or information gleaned therefrom) obtained using the sensor modules 405, 415 and 425. The sensor modules 405 are mounted in relation to the resource supply 403 so as to detect the status of the resources 430. The sensor modules 415 may similarly be mounted in relation to the equipment 410 so as to detect the status of the resources 430. The sensor modules 425 may be carried by the subject 401, or may be attached to the subject 401 (e.g., affixed as patch-module pairs as described elsewhere herein), and are similarly configured to detect the status of the resources 430.

The sensor modules 405, 415 and 425 and the feedback modules 407, 417 and 427 may be adapted for placement almost anywhere in relation to the subject 401. In human subjects, such sites include any location on the body or on equipment 410 carried by the body positioned to capture activity or the status of the equipment 410, and/or mounted relative to the resource supply 403. In illustrative embodiments, such locations for the sensor modules 405, 415 include positions directly on the resource supply 403 and/or the equipment 410 (e.g., such as adjacent a cartridge feed, magazine or the like). Various other sites for placement of the sensor modules 405, 415 and 425 are envisioned. For example, one or more of the sensor modules 405, 415 and 425 may be placed in a line of sight of the resource supply 403 and/or the equipment 410 (e.g., a cartridge feed mechanism of a weapon).

The feedback modules 407, 417 and 427 may include one or more stimulating devices intended to alert the subject 401 of a supply status of the resources 430 and/or to apprise the subject 401 of its performance. Stimulus or feedback which may be provided via one or more feedback modules 407, 417 and 427 in the resource monitoring system 400 may be in various forms, including physical stimulus (e.g., electrical, thermal, vibrational, pressure, stroking, a combination thereof, or the like), optical stimulus, acoustic stimulus, etc.

The sensor modules 405, 415 and 425, and/or the feedback modules 407, 417 and 427 of the resource monitoring system 400 may be configured for RF or other wireless and/or wired connection with one another, with other subjects (both manned and unmanned) as components of a network such as a BAN, NFC, and/or with a host device. Such wireless communication may be used to transmit or receive signals from the one or more sensors 450, 451 and 452 and/or the stimulators 470, 471 and 472.

The sensors modules 405, 415 and 425 include sensors 450, 451 and 452 which are configured to detect and collect data parameters associated with the resources 430 of the resource supply 403. Such data parameters are also referred to herein as resource tracking, resource usage or resource usage tracking signals. Consider, as an example, where the resource supply 403 is an ammunition cartridge of a weapon (e.g., equipment 410) and the resources 430 are ammunition for the weapon. As another example, the resource supply 403 may be a medical kit (e.g., that is kept in a backpack or other carrying kit providing the equipment 410) and the resources 430 may include medical supplies, medication, etc. The resource supply 403 may alternatively be a ration kit (e.g., that is kept in a backpack or other carrying kit providing the equipment 410) and the resources 430 may include food and/or water rations. The resources 430 of the resource supply 403 are to be tracked, with the sensors 450, 451 and 452 of the sensor modules 405, 415 and 425 being used to remotely collect and transmit (e.g., in real-time) data involving the status of the resources 430 of the resource supply 403 to other personnel (e.g., to a host device 145, 230, to a wearable device 302 and/or wireless gateway 340, to an AI wearable device network 348 and/or to one or more third party networks 368, etc.). Consider, as an example, resource tracking of food and/or water rations which may be used to signal that the subject 401 is (or is at risk of becoming) dehydrated (e.g., due to a lack of water), to signal that the subject 401 is (or is at risk of becoming) malnourished (e.g., due to a lack of food), etc. The real-time status of food and/or water rations may thus be used to obtain support for the subject 401 in these and other scenarios. Resource usage tracking may also be used for contextual analysis of the subject 401. For example, resource usage data may be correlated with activity of the subject 401 (e.g., what task or tasks the subject 401 is performing). This may include determining that the subject 401 is in a hostile environment or needs assistance (e.g., if resources such as ammunition, medical supplies, etc. are being expended), identifying when the subject 401 is eating or drinking, etc.

In some embodiments, the sensor modules 405, 415 and 425 and/or the feedback modules 307, 317 and 327 are designed to be subject-friendly, unobtrusive and cost-effective. In service of enabling remote evaluations of the status of the resources 430 of the resource supply 403, the sensor modules 405, 415 and 425 are configured in some embodiments to transmit data into existing inventory management, resource tracking, resource ordering and/or resource delivery databases and networks (e.g., possibly through one or more wearable devices, wireless gateways, an AI wearable device network, etc.). Such databases and networks may vary based on the environment and use case. For example, in a military use case the data collected from the sensor modules 405, 415 and 425 may be transmitted directly into one or more existing military or paramilitary information databases and/or management systems. The sensor modules 405, 415 and 425, for example, may monitor the state of the resources 430 of the resource supply 403 of the subject 401 over time in various settings including, but not limited to, battlefields or other remote deployments. Onboard sensors 450, 451 and 452 of the sensor modules 405, 415 and 425 can detect and track, for example, quantities of different ones of the resources 430 of the resource supply to determine quantities of the resources 430 remaining, quantities of the resources 430 exhausted over a given time period, etc. Such information may be used to detect additional parameters or metrics, such as a rate of consumption of different ones of the resources 430, times at which different ones of the resources 430 are expected to be depleted, etc.

Different ones of the sensor modules 405, 415 and 425 may be secured to the resource supply 403, the equipment 410 and the subject 401 via various means, including but not limited to adhesives, mechanical arrangements, straps, belts, clips, etc. The sensor modules 405, 415 and 425 are illustratively robust enough for military use, but are also extremely thin and lightweight. The sensor modules 425, for example, may be adapted for placement almost anywhere on the body of the subject 401 (e.g., such as the different locations described above with respect to the patch-module pairs of FIG. 1).

In some embodiments, the sensor modules 405, 415 and 425 and/or the feedback modules 407, 417 and 427 provide functionality for determining notification settings associated with the subject 401, and to execute or delivery notifications or feedback (e.g., such as using the stimulators 470, 471 and 472 to provide various stimulus) in accordance with the determined notification settings. The notification settings, in some embodiments, may specify the types of feedback modules 407, 417 and 427 that are accessible for delivering notifications to the subject 401 (or to military or paramilitary personnel, tactical command units, supervisors other subjects in the field or anyone associated with the subject 401). The feedback modules 407, 417 and 427 may be configured to deliver visual or audible alarms (e.g., via lights and/or sounds emitted from the stimulators 470, 471 and 472). In other embodiments, the feedback modules 407, 417 and 427 may be configured to provide various other types of feedback or stimulus as described elsewhere herein, including physical stimulus (e.g., electrical, thermal, vibrational, pressure, stroking, a combination thereof, or the like), optical stimulus, acoustic stimulus, etc. In some embodiments, notifications may be delivered to remote terminals or devices other than the feedback modules 407, 417 and 427 associated with subject 401. For example, notifications may be delivered to one or more devices associated with military or paramilitary personnel, supervisors or other subjects in the field.

A process for monitoring resource consumption and/or availability performed by or using a resource monitoring system (e.g., such as resource monitoring system 400) may include collecting resource usage tracking data from the subject 401, such as via one or more of the sensor modules 405, 415 and 425. Such resource usage tracking data may include information associated with quantities of different ones of the resources 430 that are available and/or have been expended by the subject 401 over some designated period of time. More generally, the resource usage tracking data characterizes one or more states or conditions of different ones of the resources 430.

The resource monitoring system 400 may analyze the resource usage tracking data to derive various resource parameters or metrics for the resources 430. Such parameters or metrics may include, but are not limited to, rates of consumption of the different ones of the resources 430 (e.g., possibly including trend data indicating changes in the rates of consumption of the different ones of the resources 430 over time), predicted times at which different ones of the resources 430 are expected to be depleted, etc. Such parameters or metrics may be utilized to generate feedback for delivery to the subject 401 via one or more of the feedback modules 407, 417 and 427. Such feedback may indicate to the subject 401 whether different ones of the resources 430 have been depleted, or whether different ones of the resources 430 are expected to be depleted before a time at which the subject 401 is expected to be able to replenish the resources 430. In a military scenario, for example, the subject 401 may be a soldier deployed in a remote environment on a tactical mission with an expected duration. In other scenarios, the subject 401 may be traveling through remote environments in which different resource depots are geographically dispersed. Based on tracking the location of the subject 401, or on a plan of a mission or other operation being conducted by the subject 401, a determination may be made as to whether the subject 401 will pass by or be in close proximity to one or more of such resource depots. The feedback provided to the subject 401 may direct the subject 401 to refill different ones of the resources 430 at such resource depots, to diver the subject 401 from a predicted path to reach one or more of the resource depots before different ones of the resources 430 are, or are predicted to be, depleted.

Resource usage tracking data may be collected from a group of subjects other than the subject 401, and may be analyzed to determine which subjects are expected to deplete different ones of the resources 430 at which times. Based on this information, the resource monitoring system 400 may generate recommendations or feedback to the subject 401 (or other subjects) indicating whether the subject 401 may replenish a given one of the resources 430 from another subject that has excess supply of that resource. This may include providing directions for the subject 401 (and possibly the other subject) to navigate towards one another to facilitate the exchange of resources. Feedback may also be given to initiate redistribution of one or more of the resources 430 amongst a group of related subjects including the subject 401 (e.g., a military troop or squad, a team, etc.). Resource usage tracking data from multiple subjects may also be used to compare an efficiency or performance of the subjects. For example, the rate at which different subjects including subject 401 consume different ones of the resources 430 may be used to calculate scores or other metrics characterizing efficiency or performance of those subjects at different tasks.

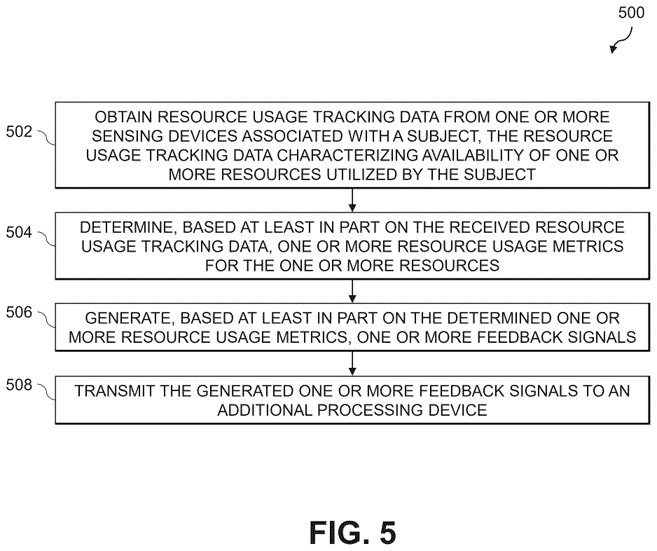

An exemplary process 500 for tracking resource usage by a subject will now be described with reference to the flow diagram of FIG. 5. It should be understood, however, that this particular process is only an example and that other types of processes for tracking resource usage by a subject may be used in other embodiments as described elsewhere herein. The process 500 includes steps 502 through 508. The process 500 may be performed, for example by various devices that are in communication with sensing devices associated with a subject such as host device 145, wearable device 302 or wireless gateway 340, etc. In step 502, resource usage tracking data is obtained from one or more sensing devices associated with a subject, the resource usage tracking data characterizing availability of one or more resources utilized by the subject. The process 500 may be performed by a host device configured to manage a network comprising the one or more sensing devices. The resource usage tracking data may be obtained in step 502 based at least on part on detecting pairing and unpairing of the one or more sensing devices with the network managed by the host device.

At least one of the one or more sensing devices may be part of one or more wearable computing devices associated with the subject. At least one of the one or more sensing devices are attached to at least one of: a resource supply device associated with at least one of the subject and a piece of equipment utilized by the subject; and the piece of equipment utilized by the subject. The piece of equipment may be configured to consume the one or more resources. In some embodiments, the one or more resources comprise ammunition, the resource supply device comprises an ammunition supply device, and the piece of equipment comprises a weapon configured to utilize the ammunition. In other embodiments, the one or more resources comprise medical supplies, the resource supply device comprises a medical supply container, and the piece of equipment comprises a medical device configured to consume the medical supplies. The one or more resources may also or alternatively comprise at least one of water and food rations.

In step 504, one or more resource usage metrics for the one or more resources are determined based at least in part on the received resource usage tracking data. One or more feedback signals are generated in step 506 based at least in part on the determined one or more resource usage metrics. The generated one or more feedback signals are transmitted to an additional processing device in step 508. The one or more resource usage metrics may comprise at least one of a quantity of the one or more resources available to the subject, a rate of consumption of the one or more resources by the subject, and a change in the rate of consumption of the one or more resources by the subject.

The at least one additional processing device may comprise a stimulating device associated with the subject, and the one or more feedback signals may instruct the stimulating device to apply a stimulus to the subject to communicate to the subject the determined one or more resource usage metrics. The at least one additional processing device may also or alternatively comprise a third-party network managing a supply of the one or more resources utilized by the subject, and the one or more feedback signals may instruct the third-party network to at least one of order an additional quantity of the one or more resources utilized by the subject and deliver the additional quantity of the one or more resources utilized by the subject.

The one or more sensing devices associated with the subject may be part of a BAN associated with the subject, the BAN comprising at least one of one or more physiologic monitoring devices and one or more location tracking devices. Determining the one or more resource usage metrics for the one or more resources in step 504 may be further based at least in part on at least one of physiologic monitoring data obtained from the one or more physiologic monitoring devices and location data obtained from the one or more location tracking devices. In some embodiments, step 504 comprises predicting a change in consumption of the one or more resources based at least in part on at least one of the physiologic monitoring data and the location data. The predicted change in the consumption of the one or more resources may be based at least in part on movement of the subject from a first location towards a second location. The second location may have at least one of different environmental conditions than the first location and different availability of resource re-supply depots. The predicted change in the consumption of the one or more resources may also or alternatively be based at least in part on detecting a change in a health of the subject from a first health state to a second health state. The generated one or more feedback signals may instruct the subject to move from a first location to a second location, the second location being associated with at least one of a resource re-supply depot and an additional subject having a quantity of the one or more resources available for transfer to the subject.

It will be appreciated that additional advantages and modifications will readily occur to those skilled in the art. Therefore, the disclosures presented herein and broader aspects thereof are not limited to the specific details and representative embodiments shown and described herein. Accordingly, many modifications, equivalents, and improvements may be included without departing from the spirit or scope of the general inventive concept as defined by the appended claims and their equivalents.

What is claimed is:

1. An apparatus comprising:
at least one processing device comprising a processor coupled to a memory;
the at least one processing device being configured:
to obtain resource usage tracking data from one or more sensing devices associated with a subject, the resource usage tracking data characterizing availability of one or more resources utilized by the subject;
to determine, based at least in part on the received resource usage tracking data, one or more resource usage metrics for the one or more resources;
to generate, based at least in part on the determined one or more resource usage metrics, one or more feedback signals; and
to transmit the generated one or more feedback signals to at least one additional processing device.

2. The apparatus of claim 1, wherein at least one of the one or more sensing devices is part of one or more wearable computing devices associated with the subject.

3. The apparatus of claim 1, wherein at least one of the one or more sensing devices is attached to at least one of:
a resource supply device associated with at least one of the subject and a piece of equipment utilized by the subject; and
the piece of equipment utilized by the subject.

4. The apparatus of claim 3, wherein the piece of equipment is configured to consume the one or more resources.

5. The apparatus of claim 3, wherein the one or more resources comprise ammunition, the resource supply device comprises an ammunition supply device, and the piece of equipment comprises a weapon configured to utilize the ammunition.

6. The apparatus of claim 3, wherein the one or more resources comprise medical supplies, the resource supply device comprises a medical supply container, and the piece of equipment comprises a medical device configured to consume the medical supplies.

7. The apparatus of claim 1, wherein the one or more resources comprise at least one of water and food rations.

8. The apparatus of claim 1, wherein the one or more resource usage metrics comprise at least one of:
a quantity of the one or more resources available to the subject;
a rate of consumption of the one or more resources by the subject; and
a change in the rate of consumption of the one or more resources by the subject.

9. The apparatus of claim 1, wherein the at least one additional processing device comprises a stimulating device associated with the subject, the one or more feedback signals instructing the stimulating device to apply a stimulus to the subject to communicate to the subject the determined one or more resource usage metrics.

10. The apparatus of claim 1, wherein the at least one additional processing device comprises a third-party network managing a supply of the one or more resources utilized by the subject, and wherein the one or more feedback signals instruct the third-party network to at least one of:
order an additional quantity of the one or more resources utilized by the subject; and
deliver the additional quantity of the one or more resources utilized by the subject.

11. The apparatus of claim 1, wherein the one or more sensing devices associated with the subject are part of a body area network associated with the subject, the body area network comprising at least one of one or more physiologic monitoring devices and one or more location tracking devices.

12. The apparatus of claim 11, wherein determining the one or more resource usage metrics for the one or more resources is further based at least in part on at least one of physiologic monitoring data obtained from the one or more physiologic monitoring devices and location data obtained from the one or more location tracking devices.

13. The apparatus of claim 12, wherein determining the one or more resource usage metrics comprises predicting a change in consumption of the one or more resources based at least in part on at least one of the physiologic monitoring data and the location data.

14. The apparatus of claim 13, wherein the predicted change in the consumption of the one or more resources is based at least in part on movement of the subject from a first location towards a second location.

15. The apparatus of claim 14, wherein the second location has at least one of:
different environmental conditions than the first location; and
different availability of resource re-supply depots.

16. The apparatus of claim 13, wherein the predicted change in the consumption of the one or more resources is based at least in part on detecting a change in a health of the subject from a first health state to a second health state.

17. The apparatus of claim 12, wherein the generated one or more feedback signals instruct the subject to move from a first location to a second location, the second location being associated with at least one of a resource re-supply depot and an additional subject having a quantity of the one or more resources available for transfer to the subject.

18. The apparatus of claim 1, wherein the at least one processing device is part of a host device configured to manage a network comprising the one or more sensing devices, and wherein the resource usage tracking data is obtained based at least in part on monitoring pairing and unpairing of the one or more sensing devices with the network managed by the host device.

19. A method comprising:
obtaining, at a host device, resource usage tracking data from one or more sensing devices associated with a subject, the resource usage tracking data characterizing availability of one or more resources utilized by the subject:
determining, at the host device based at least in part on the received resource usage tracking data, one or more resource usage metrics for the one or more resources;
generating, at the host device based at least in part on the determined one or more resource usage metrics, one or more feedback signals; and
transmitting, from the host device to at least one additional device, the generated one or more feedback signals.

20. A computer program product comprising a non-transitory processor-readable storage medium having stored therein executable program code which, when executed, causes at least one processing device:
to obtain resource usage tracking data from one or more sensing devices associated with a subject, the resource usage tracking data characterizing availability of one or more resources utilized by the subject;
to determine, based at least in part on the received resource usage tracking data, one or more resource usage metrics for the one or more resources;
to generate, based at least in part on the determined one or more resource usage metrics, one or more feedback signals; and
to transmit the generated one or more feedback signals to at least one additional processing device.
